(19) United States
(12) Patent Application Publication
(10) Pub. No.: US 2023/0017972 A1
(43) Pub. Date: Jan. 19, 2023

(54) Side-Port Injection Devices For Use With Electroporation, and Related Systems and Methods

(71) Applicant: Inovio Pharmaceuticals, Inc., Plymouth Meeting, PA (US)

(72) Inventors: Paul Fisher, Fallbrook, CA (US); Jay McCoy, Temecula, CA (US); Alison A. Generotti, Ocean City, NJ (US); Brenden Ulysses Zounes, San Diego, CA (US); Ryne Lucas Contreras, San Diego, CA (US)

(21) Appl. No.: 17/855,349

(22) Filed: Jun. 30, 2022

Related U.S. Application Data
(60) Provisional application No. 63/217,069, filed on Jun. 30, 2021.

(51) Int. Cl. A61M 5/32; A61N 1/32; A61M 5/24

(52) U.S. Cl. CPC A61M 5/3291; A61N 1/327; A61M 5/3298; A61M 5/2466

(57) ABSTRACT

An injection device for in vivo delivery of an agent includes a tubular body defining a lumen that extends along a central axis that is oriented along a longitudinal direction. A distal end of the lumen is occluded and the tubular body defines at least one side-port extending from the lumen to an outer surface of the tubular body. The at least one side-port is elongated along the outer surface of the tubular body.

### CROSS-REFERENCE TO RELATED APPLICATIONS

The present application claims the benefit of U.S. Provisional Application No. 63/217,069, filed Jun. 30, 2021, the entire contents of which are incorporated herein by this reference.

### TECHNICAL FIELD

The present invention relates to electroporation devices, and more particularly to handheld electroporation devices that include fenestrated delivery needles for delivering an injectate to tissues targeted for electroporation.

### BACKGROUND

The classical mode of administering vaccines and other pharmaceutical agents into the body tissues is by direct injection into muscle or skin tissues using a syringe and needle. Incorporating electroporative pulses of electric energy at or near the injection site is known to facilitate delivery of such vaccines or agents directly into the cells within the tissue. Such direct delivery to cells using electroporative electric pulses can have a profound clinical effect on the quality of the response of the body's metabolic and/or immune systems over that of simple syringe and needle injection. Moreover, the capability of direct delivery of agents into the cell via electroporation has enabled the effective delivery of therapeutic agents (e.g., DNA-encoded monoclonal antibodies (dMAb), expressible naked DNA encoding a polypeptide, expressible naked DNA encoding a protein, recombinant nucleic acid sequence encoding an antibody, and the like) having any number of functions, including antigenic for eliciting of immune responses, or alternatively, metabolic for affecting various biologic pathways that result in a clinical effect.

Side-port injection devices, such as fenestrated injection needles and the like, have demonstrated favorable characteristics for injecting agents into target tissue, such as intramuscular (IM) tissue. However, challenges remain with respect to providing side-port injections that disperse from the fenestrated needle in a targeted or directed manner, particularly with respect to delivering the injectate accurately and repeatably within an electroporation field created by adjacent penetrating electrodes.

### SUMMARY

According to an embodiment of the present disclosure, an injection device for in vivo delivery of an agent includes a tubular body defining a lumen that extends along a central axis that is oriented along a longitudinal direction. A distal end of the lumen is occluded and the tubular body defines at least one side-port extending from the lumen to an outer surface of the tubular body. The at least one side-port is elongated along the outer surface of the tubular body.

According to another embodiment of the present disclosure, an assembly for in vivo delivery of an agent includes an electroporation device having an electrode array that includes a plurality of needle electrodes configured for delivering one or more electroporation pulses to tissue. The assembly includes at least one injection needle that is attachable to the electroporation device in a manner extending substantially parallel with at least one of the plurality of needle electrodes. The at least one injection needle defines a lumen that extends along a central axis that is oriented along a longitudinal direction. A distal end of the lumen is occluded and the injection needle defines at least one side-port extending from the lumen to an outer surface of the injection needle. The at least one side-port is elongated along the outer surface of the injection needle.

According to an additional embodiment of the present disclosure, an electroporation system for causing in vivo reversible electroporation in cells of tissue includes an electrode array that includes a support member having a top surface and a bottom surface and defining a plurality of channels extending from the top surface to the bottom surface. A plurality of needle electrodes are coupled to the support member and extend through the plurality of channels, such that distal ends of the plurality of needle electrodes extend to a needle depth below the bottom surface of the support member. The plurality of needle electrodes are arranged in a pattern along the support member. At least some of the plurality of needle electrodes are dual-purpose injection needle electrodes that are configured to inject an agent into the tissue and are also configured to deliver one or more electroporation pulses to the tissue for causing the reversible electroporation in the cells thereof.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the features of the present application, there is shown in the drawings illustrative embodiments. It should be understood, however, that the application is not limited to the precise arrangements and instrumentalities shown. The patent or application file contains at least one drawing executed in color. Copies of this patent or patent application publication with color drawing(s) will be provided by the Office upon request and payment of the necessary fee. In the drawings:

FIG. 1A is a diagram view of an electroporation system having a hand-held electroporation device that incorporates at least one fenestrated or “side-port” injection needle, according to an embodiment of the present disclosure;

FIG. 1B is an enlarged perspective view of an electrode array of the electroporation system illustrated in FIG. 1A;

FIG. 1C is an exploded view of the electroporation device illustrated in FIG. 1A;

FIG. 1D is a sectional side view of a distal portion of the electroporation device illustrated in FIG. 1A;

FIG. 1E is a diagram view of the side-port injection needle and electrode array of the electroporation device illustrated in FIG. 1A;

FIG. 2A is a side view of a side-port injection needle, according to an embodiment of the present disclosure;

FIG. 2B is an enlarged side view of a distal region of the injection needle illustrated in FIG. 2A, showing an array of side-ports having elongated geometries;

FIG. 2C is a sectional end view of the injection needle taken along a row of side-port, along section line 2C-2C illustrated in FIG. 2B;

FIG. 2D is a sectional end view of the injection needle taken along another row of side-ports, section line 2D-2D illustrated in FIG. 2B;

FIG. 2E is an enlarged side view of a portion of the side-port array of the injection needle, showing one of the side-ports;

FIG. 2F is a sectional side view of the portion of the injection needle illustrated in FIG. 2E;

FIGS. 2G-2H are sectional side views of portions of respective injection needles having occluded distal regions, according to respective embodiments of the present disclosure;

FIG. 3 is a perspective view of a distal end of a side-port injection needle having a single, longitudinally elongated side-port, according to an embodiment of the present disclosure;

FIG. 4A is a perspective view of a distal end of a side-port injection needle having a single, laterally elongated side-port, according to an embodiment of the present disclosure;

FIG. 4B is a sectional end view of the injection needle taken along the side-port illustrated in FIG. 4A;

FIG. 5A is a perspective view of a distal portion of a side-port injection needle having a single side-port, according to an embodiment of the present disclosure;

FIG. 5B is a sectional side view of the portion of the injection needle illustrated in FIG. 5A, showing the side-port extending proximally at an oblique angle from the lumen of the injection needle;

FIG. 6 is a side view of a distal portion of a side-port injection needle having an array of circular side-ports that are arranged to approximate the geometries of the elongated side-ports illustrated in FIG. 2A, according to an embodiment of the present disclosure;

FIG. 7A is a side view of a side-port injection needle having side-ports that are arranged on a distinct circumferential portion of the needle, according to an embodiment of the present disclosure;

FIG. 7B is a sectional end view of the side-port injection needle taken along section line 7B-7B illustrated in FIG. 7A;

FIG. 8A is a side view of a distal portion of a side-port injection needle having a series of longitudinally aligned, circular side-ports;

FIG. 8B is a side view of a side-port injection needle adapted for use with a drug cartridge, according to an embodiment of the present disclosure;

FIG. 8C is a diagram view of a side-port injection needle of the present disclosure adapted for use with the handset of a CELLECTRA® 5PSP electroporation device, according to an embodiment of the present disclosure;

FIGS. 9A-9F are diagram views of distal portions of side-port injection needles showing various side-port arrays;

FIGS. 10A-10B show fluidic images of standard (bolus-type) injections in pig muscle tissue, taken at perpendicular views (FIG. 10A taken perpendicular to the direction of muscle fiber extension, and FIG. 10B taken along the direction of muscle fiber extension);

FIGS. 10C-10D show fluidic images of side-port injections in pig muscle tissue, taken at perpendicular views, using a side-port injection needle similar to that illustrated in FIGS. 2A-2F; FIG. 10C is taken perpendicular to the direction of muscle fiber extension; FIG. 10D is taken along the direction of muscle fiber extension;

FIGS. 11A-11D show fluidic images of side-port injections in pig muscle tissue comparing the effect of injection volume on fluid dispersion using a side-port injection needle similar to that illustrated in FIGS. 2A-2F; FIGS. 11A-11B show perpendicular views of a 1 mL injection and FIGS. 11C-11D show perpendicular views of a 2 mL injection (FIGS. 11A and 11C are taken perpendicular to the direction of muscle fiber extension, while FIGS. 11B and 11D are taken along the direction of muscle fiber extension);

FIG. 11E is a graph comparing dMAb expression in rabbits following side-port injections of 1 mL and 2 mL and electroporation;

FIGS. 12A-12B show test results evaluating the effect of intramuscular adipose deposits on side-port fluid dispersion; FIGS. 12A is an image showing a test setup involving an electrode array with a side-port injection needle inserted within pig muscle tissue; FIG. 12B is a fluidic image showing injectate fluid dispersion in the tissue illustrated in FIG. 12A;

FIG. 13A is a plan view of the electrode array illustrated in FIG. 1B;

FIGS. 13B-13D are diagram views showing respective example pulsing patters of the electrode array illustrated in FIG. 13A; specifically, FIG. 13B shows an example “standard” pulsing patter; FIG. 13C shows an example “star” pulsing pattern; and FIG. 13D shows an example “perimeter” pulsing pattern;

FIGS. 14A-14B show test results comparing the effects of standard injection (FIG. 14A) and side-port injection (FIG. 14B) on cellular infiltration in muscle tissue of rabbits;

FIG. 15 shows test results comparing the effects of standard injection and side-port injection and subsequent electroporation at different amperages (0.5 Amp for standard injection, 1.0 Amp for side-port injection) on cellular infiltration in muscle tissue of rabbits;

FIGS. 16A-16E show test results of an eight-week study evaluating immune responses induced by delivery of pGX3024 (a DNA plasmid) via standard injection compared to side-port injection, using different injection volumes (1 mL and 6 mL for each injection type), and employing subsequent electroporation at different amperages (0.5 Amp for all standard injections, 1.0 Amp for all side-port injection). FIG. 16A shows combined results over the eight-week study. FIGS. 16B-16E show IFNγ ELISpot data at week 0 (FIG. 16B), week 2 (FIG. 16C), week 5 (FIG. 16D), and week 8 (FIG. 16E).

FIGS. 17A-17D are graphs showing dMAb expression in rabbits (FIG. 17A), rhesus monkeys (FIG. 17B), and pigs (FIG. 17C-17D), and following standard injection and electroporation at 0.5 Amp versus side-port injection and electroporation at 1.0 Amp;

FIG. 18A is a graph showing the effect of side-port infusion length (L2) on dMAb expression in rabbits;

FIG. 18B is medical imagery showing fluid dispersion from a side-port injection;

FIG. 19 is a chart comparing the effect of side-port shape and total side-port surface area on dMAb expression in rabbits;

FIG. 20 is a chart comparing the effect of side-port shape at approximately equivalent total side-port surface areas on dMAb expression in rabbits;

FIG. 21 is a chart comparing the effect of injection rate through equivalent rectangular side-ports on dMAb expression in rabbits;

FIGS. 22A-22B are charts showing test results to identify the interaction between injection method (side-port vs standard needle) and electroporation amperage (0.5 Amp, 0.8 Amp, or 1.0 Amp pulse current with a 200 Volt maximum pulse voltage);

FIG. 23 is a chart showing the impact of plasmid concentration on side-port delivery in rabbits;

FIGS. 24A-24B show charts comparing dMAb expression in nonhuman primates following standard injection and electroporation at 0.5 Amp and side-port injection and electroporation at 1.0 Amp;

FIG. 25 is a chart showing the impact of pulse duration on dMAb expression following side-port delivery in rabbits;

FIG. 26 is a chart showing the impact of different pulse firing patterns on dMAb expression following side-port delivery in rabbits;

FIGS. 27A-27B are charts showing the impact of pulse amperages above 1.0 Amp on dMAb expression following side-port delivery;

FIG. 28 is a chart showing the impact of pulse duration on the “star” pulse pattern shown in FIG. 13C, following dMAb delivery in rabbits;

FIG. 29 is a chart showing the impact of pulse amperage on the “star” pulse pattern shown in FIG. 13C, following side-port dMAb delivery in rabbits;

FIG. 30A is a top view of an array having electroporation needles arranged in a 5×2 matrix and injection channels for receiving injection needles interspersed between the electroporation needles, according to an embodiment of the present disclosure;

FIG. 30B is a side view of the electroporation needle array illustrated in FIG. 30A;

FIG. 30C is a graph showing gene expression in rabbits following injection and electroporation with the electroporation needle array illustrated in FIGS. 30A-30B compared to other electroporation devices;

FIG. 31A is a perspective view of an array having electroporation needles arranged in a 6×4 matrix and injection channels for receiving injection needles interspersed between the electroporation needles, according to an embodiment of the present disclosure;

FIG. 31B is a side view of the array illustrated in FIG. 31A;

FIG. 31C is a bottom view of the array illustrated in FIG. 31A;

FIG. 31D is a top view of the array illustrated in FIG. 31A;

FIG. 32A is a bottom view of an array similar to the array shown in FIGS. 31A-31D but having different inter-electrode spacing, according to an embodiment of the present disclosure;

FIG. 32B is a top view of the array illustrated in FIG. 32A;

FIG. 32C is a bottom view showing calculated electric field magnitudes of the array illustrated in FIG. 32A;

FIG. 33A is a bottom view of a modular array having electroporation needles arranged in a 6×4 matrix and injection channels for receiving injection needles interspersed between the electroporation needles, according to an embodiment of the present disclosure;

FIG. 33B is a graph showing gene expression in pigs following injection and electroporation using various regions of the array illustrated in FIGS. 33A;

FIG. 33C is a graph showing gene expression following injection and electroporation using various injection volumes and various regions of the array illustrated in FIGS. 33A;

FIGS. 34A is a perspective view of an electroporation system that employs a hand-held electroporation device having an electrode array, in which the needle electrodes are dual-purpose side-port injection needles that are configured to both deliver injectate to target tissue and deliver one or more electroporative pulses to the target tissue, according to an embodiment of the present disclosure;

FIG. 34B is an enlarged perspective view of the electrode array of the hand-held electroporation device illustrated in FIG. 34A;

FIG. 34C is a perspective sectional view of an electrode array assembly of the hand-held electroporation device illustrated in FIG. 34A;

FIG. 35A is a perspective view of an electroporation system that includes an array having dual-purpose electroporation side-port needles arranged in a matrix, in which the needle electrodes are dual-purpose side-port injection needles that are configured to both deliver injectate to target tissue and deliver one or more electroporative pulses to the target tissue, according to an embodiment of the present disclosure;

FIG. 35B is a perspective view of an array assembly of the electroporation system illustrated in FIG. 35A;

FIG. 35C is a plan view showing the array assembly inserted within muscle tissue;

FIG. 36A is a bottom view of an electroporation array assembly having electroporation needles arranged in a 3×2 matrix and injection channels that are eccentrically offset from the electroporation needles, according to an embodiment of the present disclosure;

FIG. 36B is a side view of the electroporation array assembly illustrated in FIG. 36A;

FIGS. 37A-37B are fluidic images of side-port injections in pig muscle tissue taken at perpendicular views; a 3-mL dose of injectate was fractionated into three (3) separate 1-mL doses using a 3×2 matrix array having three (3) injection channels, configured similarly to the array shown in FIGS. 36A-36B;

FIGS. 37C-37D are fluidic images of a side-port injection in pig muscle tissue taken at perpendicular views using the same 3×2 matrix array used for FIGS. 37A-37B; however, in FIGS. 37C-37D, a 3-mL dose of injectate was injected using the center-most injection channel of the matrix array;

FIG. 37E is a graph comparing dMAb expression in rabbits following fractionated versus non-fractionated 3-mL side-port injections, each performed using a 3×2 matrix array similar to the array shown in FIGS. 37A-37D;

FIG. 38A is a bottom view of an electroporation array assembly having electroporation needles arranged in a 3×2 matrix and injection channels that are in-line with the rows of electroporation needles, according to an embodiment of the present disclosure;

FIG. 38B is a side view of the electroporation array assembly illustrated in FIG. 38A;

FIGS. 39A-39C are diagram views showing example pulsing patterns for the electrode array illustrated in FIGS. 38A-38B;

FIGS. 40A-40B are plan views showing the electroporation array assembly of FIG. 38A inserted within muscle tissue at parallel (FIG. 40A) and perpendicular (FIG. 40B) orientations relative to the muscle fibers; and

FIG. 40C is a set of diagram views showing calculated electric field magnitudes of an electrode row of the array illustrated in FIGS. 38A-38B at various orientations with respect to the muscle fibers.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure. Also, as used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “approximately”, “about”, and “substantially”, as used herein with respect to dimensions, angles, ratios, and other geometries, takes into account manufacturing tolerances. Further, the terms “approximately”, “about”, and “substantially” can include 10% greater than or less than the stated dimension, ratio, or angle. Further, the terms “approximately”, “about”, and “substantially” can equally apply to the specific value stated.

The term “agent”, as used herein, means a polypeptide, a polynucleotide, a small molecule, or any combination thereof. The agent may be a recombinant nucleic acid sequence encoding an antibody, a fragment thereof, a variant thereof, or a combination thereof. The agent may be a recombinant nucleic acid sequence encoding a polypeptide or protein. The agent may be formulated in water or a buffer, such as saline-sodium citrate (SSC) or phosphate-buffered saline (PBS), by way of non-limiting examples.

The term “intradermal” as used herein, means within the layer of skin that includes the epidermis (i.e., the epidermal layer, from the stratum corneum to the stratum basale) and the dermis (i.e., the dermal layer).

The term “intramuscular” as used herein, means within muscle tissue, including skeletal muscle tissue and smooth muscle tissue.

The term “adipose”, as used herein, means the layer containing adipocytes (i.e., fat cells) that reside in the subcutaneous layer.

The term “electroporation”, as used herein, means employing an electrical field within tissue that temporarily and reversibly increases the permeability and/or porosity of the cell membranes of cells in the tissue, thereby allowing an agent, for example, to be introduced into the cells. It should be appreciated that the type of electroporation disclosed herein refers to reversible electroporation (also referred to as “reversible poration”), meaning that the electroporated cell membranes (or at least a majority thereof) return to a substantially non-permeable and/or non-porous state following electroporation.

The term “electroporation field”, as used herein, means an electric field capable of electroporating cells. In instances where an electric field includes a portion that is capable of electroporating cells and another portion that is incapable of electroporating cells, the “electroporation field” refers specifically to that portion of the electric field that is capable of electroporating cells. Thus, an electroporation field can be a subset of an electric field.

The embodiments disclosed herein pertain to fenestrated delivery needles and electroporation devices that employ one or more such fenestrated delivery needles. Such delivery needles include at least one, and preferably a plurality of, apertures defined in the sides of the needle body. These apertures, also referred to herein as “side-ports”, are in fluid communication with a lumen of the needle. Prior art fenestrated delivery needles include generally circular side-ports. The side-port delivery needles described herein are adapted to enhance injectate fluid dispersion in tissue, such as muscle tissue, particularly by increasing the fluid dispersion along one or more directions that extend radially outwardly from the delivery needle, as opposed to distally from the distal end of the delivery needle. This is particularly beneficial for localizing the injectate within an electroporation field within the tissue, such as an electroporation field created by one or more elongate needle electrodes extending parallel with the delivery needle. By increasing radial dispersion and reducing distal dispersion of the injectate, the side-port delivery needles described herein can better align or co-localize the injectate with the electroporation field within the tissue, resulting in increased transfection of agents carried by the injectate into cells of the tissue. Many of the embodiments disclosed herein have demonstrated particularly enhanced fluid dispersion characteristics in muscle tissue. While desiring not to be bound by any particular theory, one reason the inventors believe that embodiments herein demonstrate such favorable fluid dispersion characteristics in muscle tissue is because the side-ports have been better adapted to direct the ejected fluid along directions running parallel to the directions along which the muscle fibers extend.

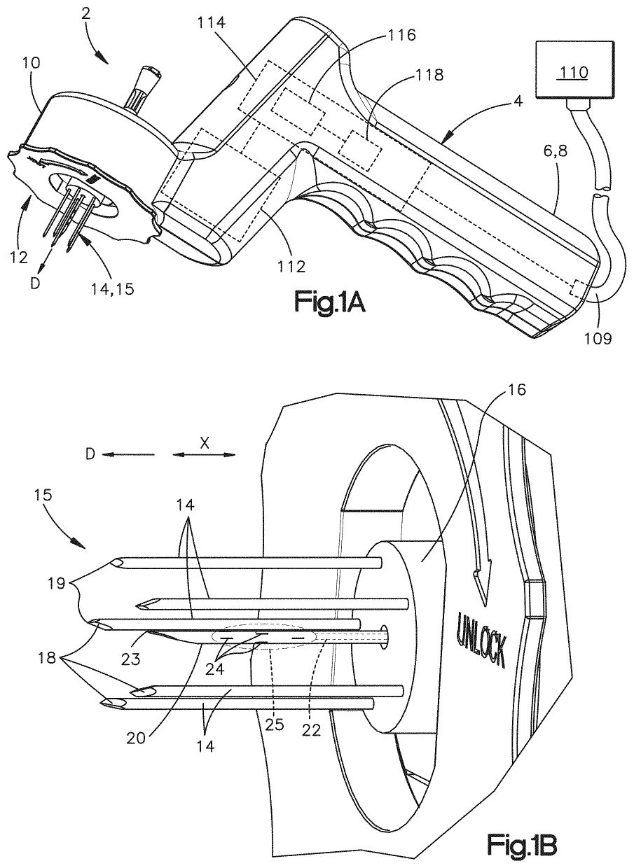

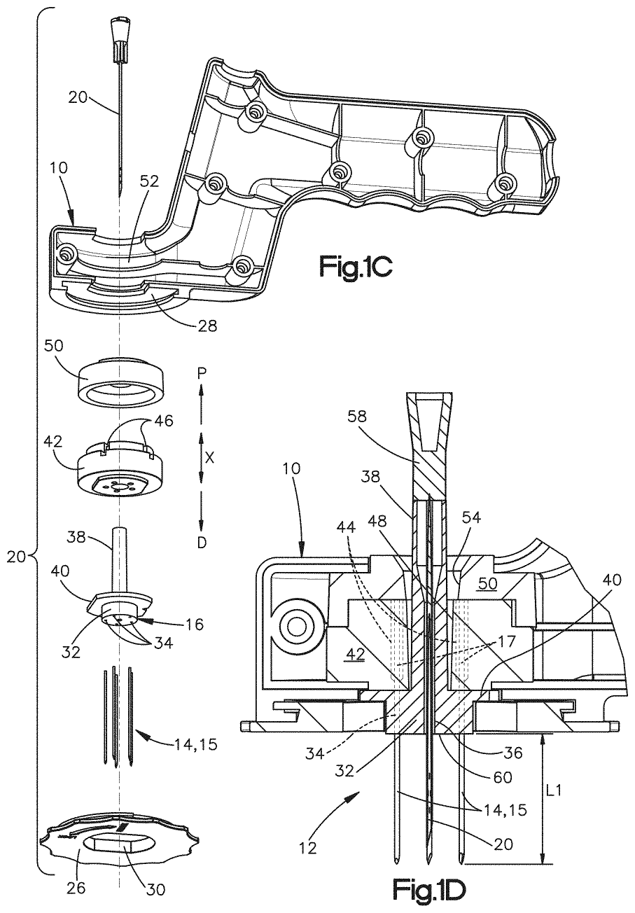

Referring to FIGS. 1A-1B, an electroporation system 2 according to an exemplary embodiment of the present disclosure includes a hand-held electroporation device 4 that includes a housing 6. The hand-held electroporation device 4 can also be referred to as an “applicator” 4. The electroporation device 4 includes a handle 8 and a mounting portion 10 (also referred to herein as a “mounting head” or “applicator head” 10) extending distally from the handle 8. The handle 8 and applicator head 10 can be defined by the housing 6. The applicator head 10 can carry an array assembly 12 that includes one or more electrodes 14, such as a plurality of electrodes 14 in a spatial arrangement, which arrangement can be referred to as an “electrode array” 15. The electrodes 14 extend from a support member 16 in a distal direction D. The electrodes 14 of this embodiment are penetrating electrodes that have distal tips 18 configured to penetrate tissue, particularly for penetrating through dermal tissue and into muscle tissue. One or more and up to all of the distal tips 18 can be a trocar tip having planar surfaces that converge to a point at a distal end 19 of the electrode 14, by way of a non-limiting example.

The electrodes 14 are configured to deliver one or more pulses of electrical energy to cells of the target tissue, specifically for reversibly electroporating the cells. The device 4 includes circuitry for providing electrical communication between the electrodes 14 and an energy source 110. As shown, the circuitry can be configured to connect with one or more cables 109 configured to couple with an energy source 110 located remote from the hand-held electroporation device 4, such as a power generator. Additionally or alternatively, the circuitry can be configured to connect with an on-board energy source, such as a battery unit disposed within the housing 6.

The energy source 110 can be in electrical communication with a pulse generator 112, such as a waveform generator, for generating and transmitting an electric signal in the form of one or more electrical pulses having particular electrical parameters to the electrodes 14 for electroporating cells within the tissue. Such electrical parameters include electrical potential (voltage), electric current type (alternating current (AC) or direct current (DC)), electric current magnitude (amperage), pulse duration, pulse quantity (i.e., the number of pulses delivered), and time interval or “delay” between pulses (in multi-pulse deliveries). The pulse generator 112 can include a waveform logger for recording the electrical parameters of the pulse(s) delivered. The pulse generator 112 can be in electrical communication with a control unit 114 (also referred to herein as a “controller”), which can include a processor 116 configured to control operation of the electroporation system 2, including operation of the pulse generator 112. The processor 116 can be in electronic communication with computer memory 118, and can be configured to execute software and/or firmware including one or more algorithms for controlling operation of the system 2.

The processor 116 can be in electrical communication with a user interface, which can be located on the device 4 or remote from the device 4. The user interface can include a display for presenting information relating to operation of the system 2 and inputs, such as a keypad or touch-screen, that allow a physician to input information, such as commands, relating to operation of the system 2. It should be appreciated that the interface can be a computer interface, such as a table-top computer or laptop computer, or a hand-held electronic device, such as a smart-phone or the like.

The applicator head 10 includes a fluid delivery device that includes an elongate tubular member, which in the embodiments disclosure herein is an injection needle 20, configured to deliver an injectate to a target region of tissue. As shown in FIG. 1B, the injection needle 20 has a lumen 22 that extends along a longitudinal direction X and at least one aperture or “side-port” 24 in fluid communication with the lumen 22. As shown, the injection needle 20 can include a plurality of side-ports 24 arranged in a side-port pattern or “array” 25. A distal end 23 of the injection needle 20 is preferably occluded so that, during injection, substantially all of the injected agent exits the lumen 22 out the side-ports 24. The side-ports 24 can have various geometries and can be arranged according to various port array 25 patterns, as described in more detail below. The side-port array 25 is also referred to herein as the “port array” 25. The injection needle 20 can be centrally located in the electrode array 15, as shown. This arrangement, in combination with the side-ports 24, can facilitate an injection fluid dispersion that is co-localized with the electroporation field created in the tissue by the electrodes 14. It should be appreciated that, in other embodiments, the injection needle 20 need not be centrally located in the electrode array 15. In such other embodiments, co-localization of injection fluid dispersion can be achieved by other parameters, as discussed in more detail below.

Referring now to FIGS. 1C-1D, the array assembly 12 can include one or more mounting members for mounting the electrode array 15 to the applicator head 10. For example, the array assembly 12 can include a distal mounting member 26 configured to couple with, in interlocking fashion, a complimentary distal mounting formation 28 of the applicator head 10. The distal mounting member 26 can thus also be referred to an array-locking member 26. The distal mounting member 26 can define a central aperture 30 through which the electrodes 14 extend. The support member 16 can include a hub 32, which can define a plurality of electrode apertures 34, through which the electrodes 14 can extend, respectively. In this manner, the spacing of the electrode apertures 34 in the hub 32 can define the pattern of the electrode array 15. The support member 16 defines an injection channel 36, through which the injection needle 20 can extend. The injection channel 36 can be centrally located with respect to the electrode apertures 34, although other arrangements are within the scope of the present disclosure. The support member 16 can also include an elongated proximal portion 38 (also referred to herein as a “chimney” or “riser”) that extends the injection channel 36 from the hub 32 in a proximal direction P opposite the distal direction D. It should be appreciated that the proximal and distal directions P, D are each mono-directional and extend along the longitudinal direction X, which is bi-directional. The support member 16 can also include a flange 40 located intermediate the hub 32 and the chimney 38 along the longitudinal direction X. The flange 40 can be configured to abut a proximal surface of the distal mounting member 26 when the array assembly 12 is in an assembled configuration and coupled to the applicator head 10 (FIG. 1D).

The array assembly 12 can include an intermediate mounting member 42 that defines a plurality of sockets 44 arranged correspondingly with the electrode apertures 34 of the support member 16. The sockets 44 are configured to receive proximal ends 17 of the electrodes 14. The sockets 44 are configured to provide electrical communication between the pulse generator 112 and the electrodes 14. For example, the sockets 44 can be in open communication with respective openings 46 in the intermediate mounting member 42 that allow passage for electrical leads that extend between the proximal ends 17 of the electrodes 14 and the pulse generator 112. The intermediate mounting member 42 of the present embodiment also defines an injection channel 48 that is in alignment with the injection channel 36 of the support member 16 and through which the chimney 38 can extend.

The array assembly 12 can include a proximal mounting member 50 or “cap” that is configured to couple with the intermediate mounting member 42, preferably in a manner covering the sockets 44. The cap 50 can also be configured to couple with a complimentary proximal mounting formation 52 of the applicator head 10. The cap 50 also defines an injection channel 54 configured to be in alignment with injection channels 36 and 48 when the array assembly 12 is in the assembled configuration. The injection channel 54 of the cap 50 is preferably configured so that the chimney 38 can extend therethrough. As shown in FIG. 1D, the chimney 38 can protrude proximally from the applicator head 10 when in the assembled configuration. A distal end 56 of the chimney 38 can be configured to mount with a connection member 58 (also referred to herein as a “connector”) attached to the injection needle 20. The connector 58 is configured to couple with a reservoir of the injectate, such as a syringe, a single-dose cartridge, and the like. As shown, the connector 58 can be a Luer-type connector, although other connector types and designs are within the scope of the present embodiments.

In some embodiments, the electroporation system 2 can employ the CELLECTRA® 2000 system, which has an external, battery powered pulse generator 112 (i.e., the CELLECTRA® Pulse Generator) that is connected via cable to the hand-held electroporation device 4 (i.e., the CELLECTRA® 5P-IM Applicator). The applicator head 10 of the electroporation device 4 is configured to couple with the CELLECTRA® 5P-IM Array, a sterile disposable array assembly 12 having five stainless steel needle electrodes 14. The side-port injection needle 20 can be pre-packaged with the CELLECTRA® 5P-IM Array. It should be appreciated that the CELLECTRA® products and components described above are produced by Inovio Pharmaceuticals, Inc., headquartered in Plymouth Meeting, Pa., United States.

Referring now to FIGS. 1D-1E, The applicator head 10 and/or the array assembly 12 is preferably configured to control a maximum depth L1 at which the electrodes 14 penetrate the surface of the subject's skin. This depth L1, also referred to herein as “penetration depth” or “electrode needle depth,” can be governed by a contact or “stop” surface 60 of the array assembly 12 that is configured to abut the subject's skin and halt further advancement of the electrodes 14 into the tissue. As shown, the stop surface 60 can be defined by a distal surface of the support member 16, by way of a non-limiting example. As shown in FIG. 1E, the injection needle 20 defines an infusion length L2, measured from a proximal end of the port array 25 to a distal end of the port array 25. The array assembly 12 also defines an infusion depth L3, measured from the stop surface 60 to the proximal end of the port array 25, and a distal infusion depth L4, measured from the stop surface 60 to the distal end of the port array 25. The injection needle 20 also defines a distal stand-off distance L5, measured between the distal end of the port array 25 to the distal end 23 of the injection needle 20. The electrodes 14 and the injection needle 20 are preferably cooperatively configured to co-localize the injectate with the electroporation field within target tissue. As shown, the electrode penetration depth L1 can be set, for example, such that the distal end of the port array 25 is proximally spaced from the distal ends 19 of the electrodes 14 at an infusion-electrode offset distance L6, as described in more detail below.

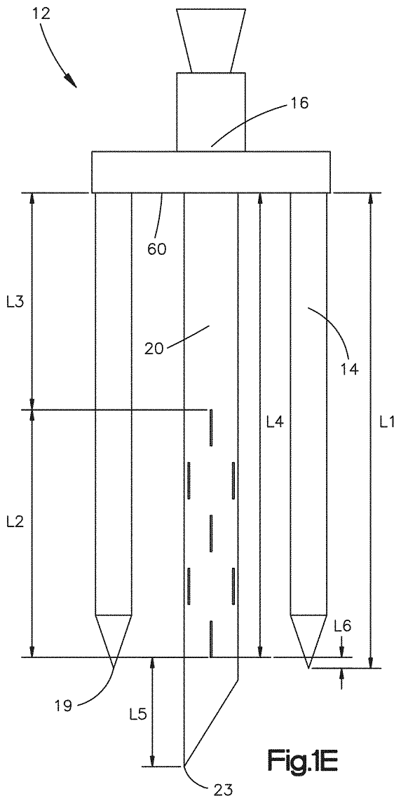

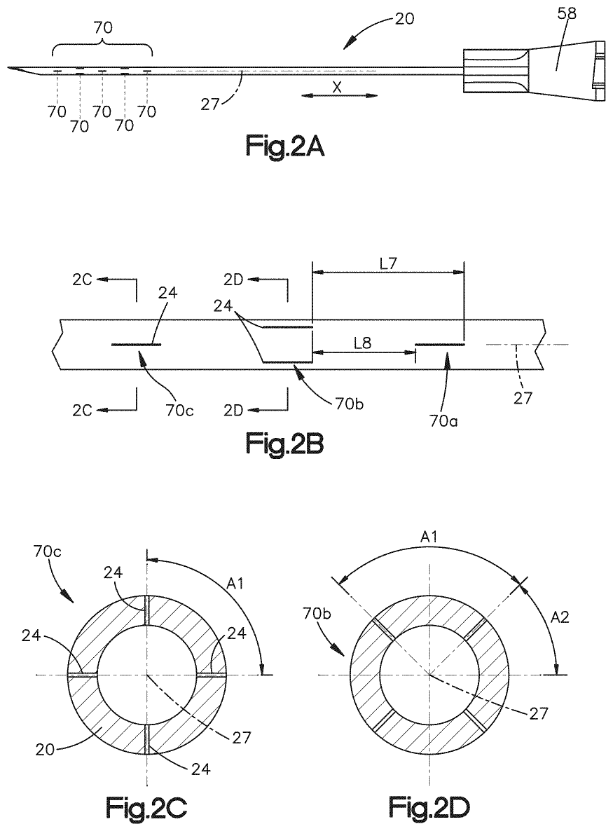

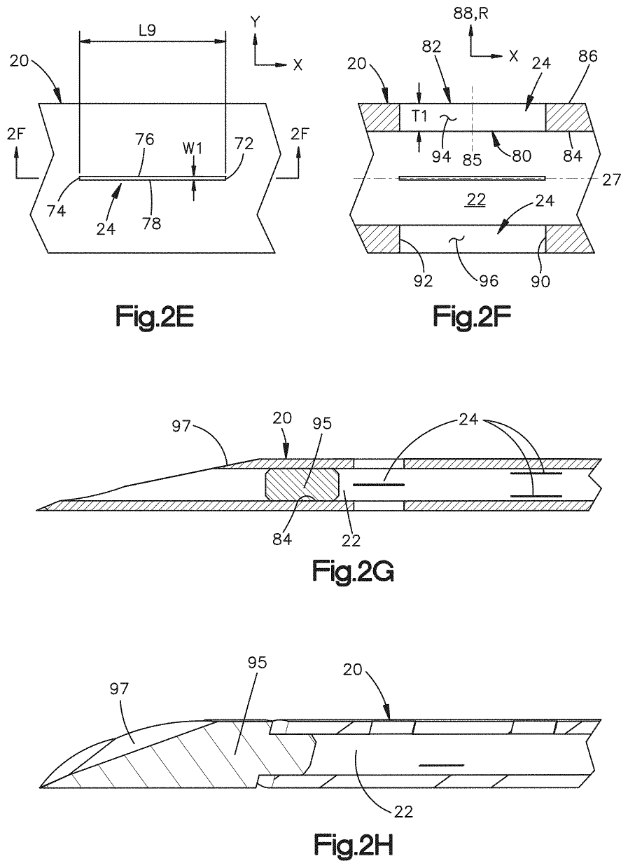

Referring now to FIGS. 2A-2F, examples of side-port 24 geometries and arrays 25 (patterns) will now be described. The side-ports 24 of the array 25 can be arranged into rows 70 that are longitudinally spaced from each (i.e., spaced from each other along the longitudinal direction X). In this illustrated example, the port array 25 has five (5) rows 70 longitudinally offset from one another, and each row 70 has four (4) side-ports 24, giving the port array 25 a total of twenty (20) side-ports 24. The illustrated port array 25 can be characterized as a “5×4” array 25. (i.e., 5 rows×4 ports per row). As shown in FIG. 2B, adjacent rows 70, such as a first row 70a and an adjacent second row 70b, can be spaced from each other at a row offset distance L7. The port array 25 can also define an inter-row distance L8 measured between adjacent rows 70. The rows 70 of the array 25 can be evenly spaced along the longitudinal direction X, according to one or both of row offset distance L7 and inter-row distance L8. In other embodiment, the rows 70 need not be evenly spaced from each other along the longitudinal direction X.

One or more of the rows 70 can also be angularly offset from at least one other row 70 about a central axis 27 of the injection needle 20. For example, side-ports 24 in adjacent rows 70 can be angularly offset from one another, such as in an angularly staggered fashion along the longitudinal direction X. As shown in FIGS. 2C-2D, each row 70 can include four (4) side-ports 24, which can be spaced from each other at even spacing angles A1, which in this example are about 90 degrees, as measured about the central axis 27. The side-ports 24 of adjacent rows 70, such as the depicted third and second rows 70c,b, can be angularly offset from each other at an offset angle A2. In the illustrated example, the offset angle A2 is about 45 degrees.

As mentioned above, the rows 70 can be angularly staggered, such that the side-ports 24 of alternating rows (e.g., the first and third rows 70a,c) are angularly aligned, and the side-ports 24 of other alternating rows (e.g., the second row 70b and a fourth row) are angularly aligned, while the rows 70 that are adjacent each other are angularly offset at angle A2, by way of a non-limiting example. The foregoing example can be referred to as “two-level” angular staggering. In other embodiments, the rows 70 of side-ports 24 can be arranged according to three-level angular, in which a first and fourth row 70 can be angularly aligned, a second and fifth row 70 can be angularly aligned, and a third and sixth row can be angularly aligned, and so forth. It should be appreciated that, in further embodiments, the rows 70 of side-ports 24 can employ four-level, five-level, six-level, seven-level, or greater than seven-level staggering. In yet other embodiments, that rows 70 of side-ports 24 can be arranged such that the each row 70 is angularly offset from every other row 70. As shown, the port array 25 can span substantially an entire circumference of the injection needle 20. In other embodiments, the port array 25 can span less than an entire circumference of the injection needle 20, examples of which are described in more detail below.

Referring now to FIGS. 2E-2F, one or more and up to all of the side-ports 24 can be elongated, such as along the longitudinal direction X. For example, any of the side-ports 24 can be elongated between a first port end 72 and an opposed second port end 74. Such elongated side-ports 24 can also define a first side 76 and an opposed second side 78. A length L9 of the side-port 24 is measured between the first and second port ends 72, 74. A width W1 of the side-port 24 is measured along a lateral direction Y that is substantially perpendicular to the longitudinal direction X. For such elongated side-ports 24, the length L9 is greater than the width W1 by a port elongation factor (i.e., L9/W1), which can be in a range of about 1.00 to about 100, and more particularly in a range of about 20 to about 60, and more particularly in a range of about 35 to about 40. The elongated side-ports 24 described herein can be characterized as having a slot-like geometry. Thus, each elongated side-port 24 can also be referred to as a “slot.”

Each side-port 24 extends from an inner opening 80 to an on outer opening 82 along a port axis 85. The inner opening 80 interfaces with an interior surface 84 of the injection needle 20 that defines lumen 22. The outer opening 82 interfaces with an outer surface 86 of the injection needle 20. The side-port 24 defines a median flow distance T1, which can be measured from the interior surface 84 to the outer surface 86 along a port flow direction 88 oriented along the port axis 85. In the illustrated example, the port axis 85 (and thus the port flow direction 88) extend along a radial direction R that extends perpendicularly from the central axis 27 of the injection needle 20.

The geometry of the side-port 24 can be further defined by first and second endwalls 90, 92, at the first and second port ends 72, 74, respectively, and first and second sidewalls, at the first and second sides 76, 78, respectively. As shown, the endwalls 90, 92 and sidewalls 94, 96 can each extend between the interior and outer surfaces 84, 86 of the injection needle 20 along the port flow direction 88, which in the illustrated example is along the radial direction R (i.e., perpendicular to the central axis 27). It should be appreciated, however, that other endwall 90, 92 and/or sidewall 94, 96 geometries are within the scope of the present disclosure. For example, any of the endwalls 90, 92 and/or sidewalls 94, 96 can be oriented oblique to the radial direction R. Similarly, the port axis 85 and port flow direction 88 can be angularly offset from the radial direction R, such as in a manner having a directional component along the longitudinal direction X, by way of a non-limiting example, and as described in more detail below. Moreover, any of the endwalls 90, 92 and/or sidewalls 94, 96 can define one or more relief surfaces, such as bevels, chamfers, and the like, which can be located at an interface with the interior surface 84 and/or the outer surface 86, and which can be configured to provide favorable flow characteristics of the injectate traveling through the respective side-port 24.

As shown in FIG. 2E, the ends 72, 74 of the side-ports 24 can be substantially perpendicular to the sides 76, 78, providing the side-ports 24 with a rectangular geometry. In other embodiments, one or both of the ends 72, 74 can be rounded. In further embodiments, the side-ports 24 can be elliptically elongated, helically elongated, or elongated according to other geometries. Additionally or alternatively, the side-ports 24 can have widths W1 that are greater at their ends 72, 74 than at intermediate portions of their sides 74, 76 (i.e., akin to a “dog bone” shape and the like).

Referring now to FIG. 2G, the distal end of the injection needle 20 is preferably occluded, as mentioned above, so that the injectate is forced from the lumen 22 through the side-ports 24. As shown, the occlusion can be provided by a plug 95 inserted into the distal opening of the injection needle 20 and sealed therein. The plug 95 can be sealed within the lumen 22 at a location proximal of the bevel 97. The plug 95 can be constructed of stainless steel or similar bio-compatible material found in hypodermic needles, by way of non-limiting examples. The plug 95 can be welded to the distal region of the interior surface 84, such as by laser welding, for example. Alternatively, the plug 95 can be constructed of a polymeric material and can be bonded within the lumen 22 via an adhesive. In yet other embodiments, as shown in FIG. 2H, the bevel 97 can be formed on the plug 95, which can be constructed or stainless steel and the like, and can be inserted and welded to the distal end of the lumen 22.

It should be appreciated that the side-ports 24 can have various geometries and can be arranged according to various port array 25 patterns. For example, the side-ports 24 can be rectangularly elongated and arranged into angularly staggered rows, such as the 3×4 array 25 shown in FIGS. 1A-D, the 5×4 array shown in FIGS. 2A-2E, or other such array patterns. It should be appreciated that the array 25 configuration can range from 1×1 (i.e., an array 25 consisting of a single side-port 24) 1×2, 1×3, 1×4, 1×5, 1×6, 1×7, 1×8, 1×9, 1×10, 1×11, 1×12, 1×12+ (i.e., one (1) row having more than twelve (12) side-ports 24), 2×1, 2×2, 2×3, 2×4, 2×5, 2×6, 2×7, 2×8, 2×9, 2×10, 2×11, 2×12, 2×12+, 3×1, 3×2, 3×3, 3×4, 3×5, 3×6, 3×7, 3×8, 3×9, 3×10, 3×11, 3×12, 3×12+, 4×1, 4×2, 4×3, 4×4, 4×5, 4×6, 4×7, 4×8, 4×9, 4×10, 4×11, 4×12, 4×12+, 5×1, 5×2, 5×3, 5×4, 5×5, 5×6, 5×7, 5×8, 5×9, 5×10, 5×11, 5×12, 5×12+, 6×1, 6×2, 6×3, 6×4, 6×5, 6×6, 6×7, 6×8, 6×9, 6×10, 6×11, 6×12, 6×12+, 7×1, 7×2, 7×3, 7×4, 7×5, 7×6, 7×7, 7×8, 7×9, 7×10, 7×11, 7×12, 7×12+, 8×1, 8×2, 8×3, 8×4, 8×5, 8×6, 8×7, 8×8, 8×9, 8×10, 8×11, 8×12, 8×12+, 9×1, 9×2, 9×3, 9×4, 9×5, 9×6, 9×7, 9×8, 9×9, 9×10, 9×11, 9×12, 9×12+, 10×1, 10×2, 10×3, 10×4, 10×5, 10×6, 10×7, 10×8, 10×9, 10×10, 10×11, 10×12, 10×12+, 11×1, 11×2, 11×3, 11×4, 11×5, 11×6, 11×7, 11×8, 11×9, 11×10, 11×11, 11×12, 11×12+, 12×1, 12×2, 12×3, 12×4, 12×5, 12×6, 12×7, 12×8, 12×9, 12×10, 12×11, 12×12, 12×12+, 12+×1 (i.e., more than twelve (12) rows 70 each having one (1) side-port 24), 12+×2, 12+×3, 12+×4, 12+×5, 12+×6, 12+×7, 12+×8, 12+×9, 12+×10, 12+×11, 12+×12, and 12+×12+, by way of non-limiting examples. It should also be appreciated that the array 25 configuration can have one or more rows 70 having a different quantify of side-ports 24 than those of at least one other row 70. It should further be appreciated that the side-ports 24 within an array 25 can have different geometries.

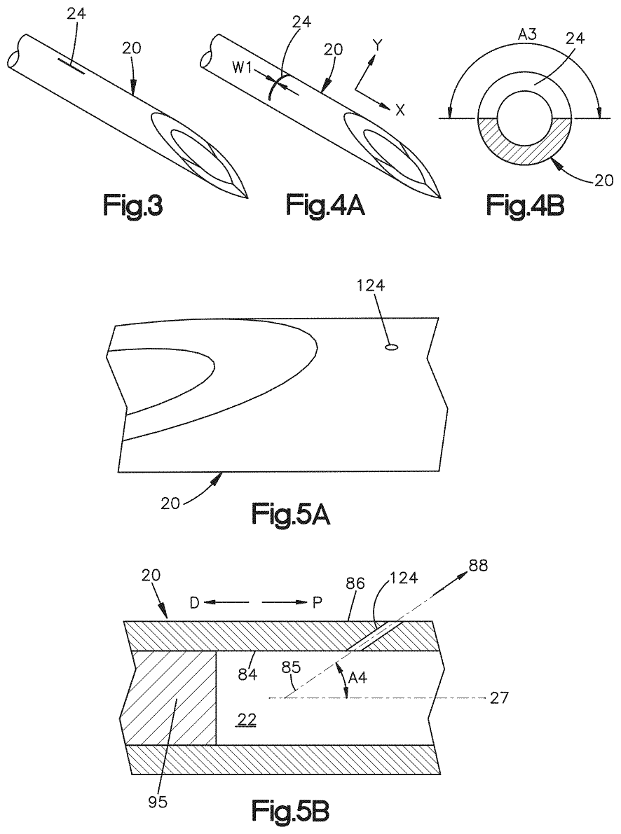

Referring now to FIGS. 3-9F, additional example side-port 24 configurations will be described.

As shown in FIG. 3, an injection needle 20 can have a single, longitudinally elongated side-port 24, which can have a geometry similar to that described above with reference to FIGS. 2E-2F.

As shown in FIGS. 4A-4B, an injection needle 20 can have a single side-port 24 that is elongated along a direction that is offset from the longitudinal direction X. As shown, the single side-port 24 of the present embodiment can be elongated along the lateral direction Y, although other offset elongation directions are within the scope of the present disclosure. The side-port 24 of the present example can define an angular span A3 in a range of about 5 degrees to about 200 degrees, and more particularly in a range of about 40 degrees to about 190 degrees, and more particularly in a range of about 150 degrees to about 180 degrees. The side-port 24 can define a width W1 measured along the longitudinal direction X within the ranges described above with reference to FIG. 2E.

As shown in FIGS. 5A-5B, an injection needle 20 can have a single side-port 124, which, in this example, has a circular shape. The side-port 124 can extend from the lumen 22 (i.e., from the interior surface 84) along a port axis 85 that is oriented at an oblique angle A4 with respect to the central axis 27 of the injection needle 20. In this manner, the side-port 124 provides a port flow direction 88 having a longitudinal directional component. In this particular example, the port flow direction 88 has a directional component in the proximal direction P. Thus, the illustrated side-port 124 is configured to eject fluid “upward” or toward a shallower depth in the tissue, which can be beneficial in some embodiments for co-localizing the injectate fluid dispersion with the electroporation field. It should be appreciated that, in other embodiments, the side-port 124 can be angled so as to provide a flow direction 88 having a directional component in the distal direction D. In further embodiments, a port array 25 can have various side-ports 24, 124 that provide various flow directions 88, including those having directional components in the proximal and/or distal directions P, D and/or those oriented substantially along the radial direction R.

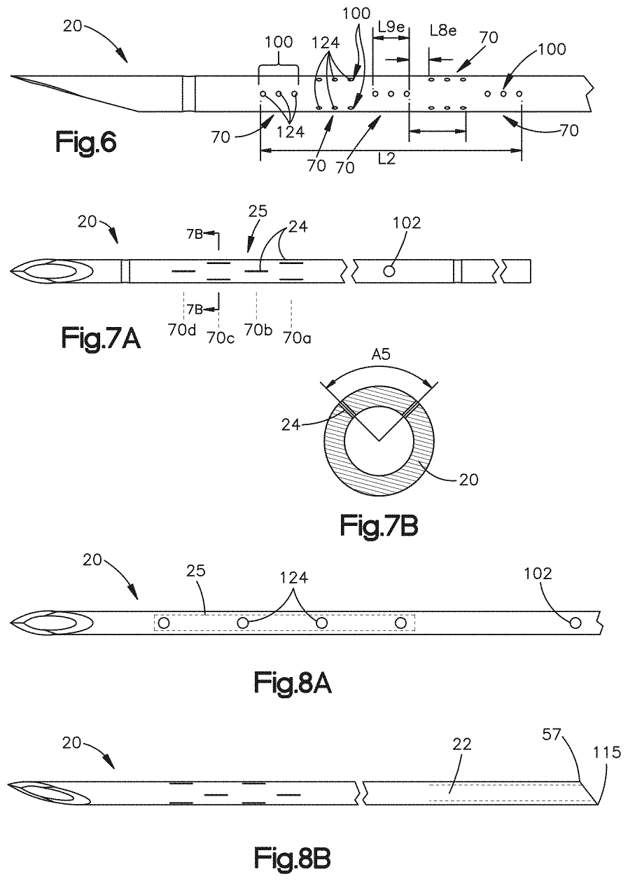

As shown in FIG. 6, an injection needle 20 can have a port array 25 that includes multiple groups 100 of side-ports 124, such that the side-ports 124 in each group 100 are aligned in a manner generally approximating the elongated, slot-type side-ports 24 described above with reference to FIGS. 1A-2F. For example, each group 100 can define a group length L9e that is within the ranges described above for the length L9 of the elongated side-ports 24 (FIG. 2E). The individual side-ports 124 of the present embodiment can be circular and can have a radius in a range of about 0.020 mm to about 0.100 mm, and more particularly in a range of about 0.025 mm to about 0.075 mm, and more particularly in a range of about 0.045 mm to about 0.055 mm. As shown, each group 100 can include three (3) side-ports 124, although in other embodiments each group 100 can include from two (2) to eight (8) side-ports 124. The groups 100 of side-ports 124 can be arranged in an array 25 pattern having rows 70, which can be angularly staggered, similar to the array 25 patterns described above with reference to FIGS. 1A-2F. Accordingly, in similar fashion, adjacent rows 70 of the present embodiment can be spaced relative to each other at an effective row offset distance L7e, and the port array 25 can employ an effective inter-row distance L8e measured between adjacent rows 70. It should be appreciated that the groups 100 can also employ the spacing angles A1 and the rows 70 can employ the offset angles A2 described above. The specific array depicted can be characterized as a 5×4×3 array (i.e., five (5) rows, each row having four (4) groups, each group having three ports). It should be appreciated that the present embodiment can have various array patterns, ranging from 1×1×1 to 12×12×12 or greater. The port array 25 of the present embodiment can define an infusion length L2 within the ranges described above.

Referring now to FIGS. 7A-7B, an injection needle 20 can have a port array 25 with elongate, slot-like side-ports 24, similar to the port arrays 25 described above with reference to FIGS. 1A-2F. However, in the present embodiment, the port array 25 can span less than an entire circumference of the injection needle 20. For example, the port array 25 can have four (4) rows 70a-d, such that first and third rows 70a,c each have two (2) side-ports 24 and the second and fourth rows 70b,d each have a single side-port 24. The first and third rows 70a,c can be angularly aligned with each other, and the second and fourth rows 70b,d can be angularly aligned with each other. In this manner, as shown in FIG. 7B, the port array 25 can define an angular span A5 that is less than the entire circumference of the injection needle 20. The angular span A5 of such embodiments can be in a range from about 5 degrees to about 270 degrees, and more particularly in a range of about 180 degrees to about 30 degrees, and more particularly in a range of about 60 degrees to about 120 degrees. It should be appreciated that the angular span A5 of the presently illustrated port array 25 can be approximately equivalent to the angular spacing between the side-ports 24 in the first and third rows 70a,c. The port array 25 of the present embodiment, and similar port arrays 25 having a limited angular span A5, can be particularly beneficial for providing a directionally controlled injectate fluid dispersion in target tissue. Thus, such port arrays 25 can be referred to as “directed” port arrays 25.

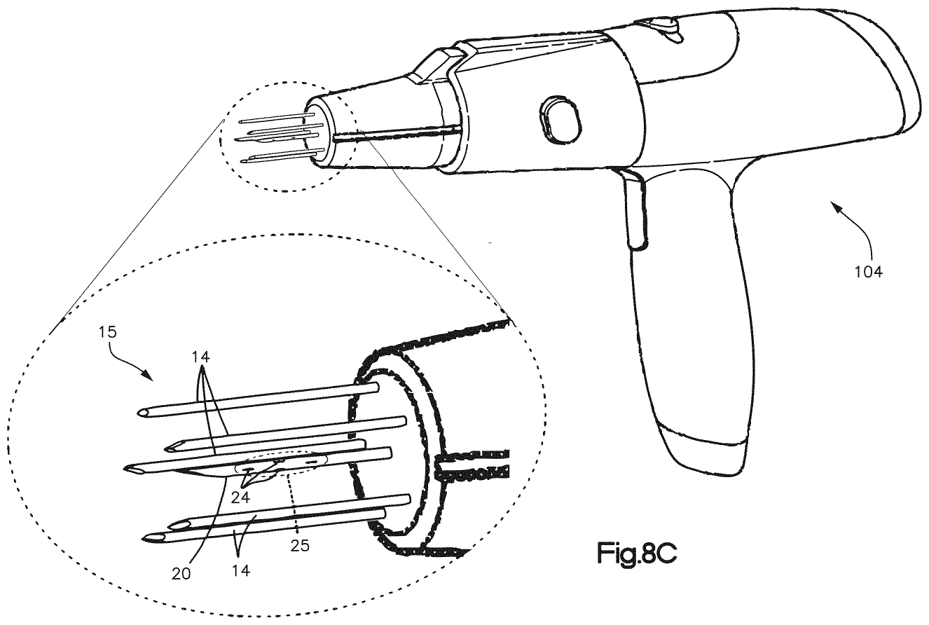

Referring now to FIG. 8A, another example of a directed-array injection needle 20 has a port array 25 that includes a plurality of side-ports 124 that are arranged in a single series, such that all of the side-ports 124 are longitudinally aligned with each other. As shown, the side-ports 124 of the present embodiment can be circular side-ports 124, although in other embodiments any of the other side-port shapes and geometries described above can be employed in a similar, single-series fashion.

It should be appreciated that directed-array injection needles 20, such as those shown in FIGS. 7A-8A, can be particularly useful when employed in an injection assembly having a plurality of such directed-array injection needles 20 that are oriented so that their angular spans A5 overlap within a target volume of tissue, such as a target volume intermediate the directed-array injection needles 20. In such multi-injection needle 20 embodiments, the injection needles 20 can be configured for connection to a manifold for controlling fluid flow to each of the injection needles 20 in the injection assembly, including simultaneous fluid flow to each of the injection needles 20. In such embodiments (and yet other embodiments), one or more and up to all of the injection needles 20 in the injection assembly can optionally include a fluid injection side-port 102 located proximally (i.e., upstream) from the port array 25, as shown in FIGS. 7A and 8A.

Referring now to FIG. 8B, another example of a side-portion injection needle 20 can be configured for use with a drug cartridge, such as a single-dose injection cartridge, by way of a non-limiting example. In such embodiments, a proximal end 57 of the injection needle 20 can define a penetrating formation, such as a proximal bevel 115, configured to penetrate a distal septum of the drug cartridge, thereby placing the lumen 22 of the injection needle 20 in fluid communication with the injectate contained within the drug cartridge. The side-port injection needle 20 can also be configured for use with a retractable shroud configured to retract in a manner exposing the side-port injection needle, such as during injection, and further configured to extend and lock in place in a manner covering the injection needle 20 after use, such as after a single use injection. For example, referring now to FIG. 8C, the side-port injection needle 20 can be configured for use with the handset 104 of the CELLECTRA® 5PSP electroporation device, which is produced by Inovio Pharmaceuticals, Inc. and is further described in U.S. Patent Publication No. 2019/0009084, published Jan. 10, 2019, entitled “ELECTROPORATION DEVICE WITH DETACHABLE NEEDLE ARRAY WITH LOCK-OUT SYSTEM,” the entire disclosure of which is hereby incorporated by reference. It should be appreciated that the side-port injection needles 20 described herein can be configured for use with numerous types of electroporation devices. Furthermore, the specific electroporation devises described herein are provided as non-limiting examples of electroporation devices that can employ the side-port injection needles 20.

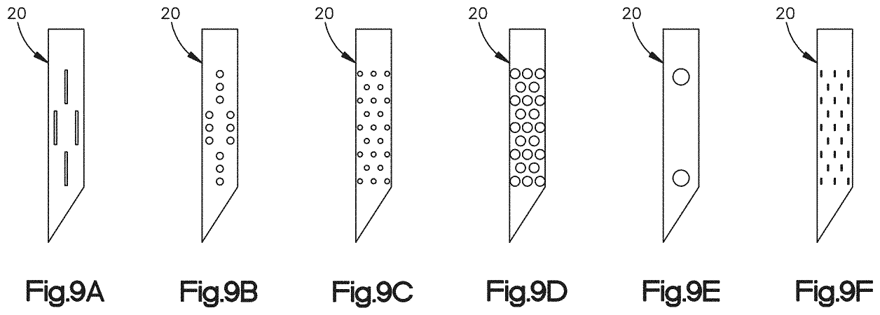

Referring now to FIGS. 9A-9F, additional non-limiting examples of port arrays 25 will now be described.

As shown in FIG. 9A, an example port array 25 includes elongated side-ports 24 arranged in three (3) rows 70 with four (4) ports per row 70 (i.e., a 3×4 array having a total of twelve (12) side-ports) that provides an angular span of 360 degrees (i.e., the entire circumference of the injection needle 20). Each row 70 has spacing angles A1 of about 90 degrees, and the middle row 70 is angularly offset at an angle A2 of about 45 degrees. Each side-port 24 has a length L9 of about 0.8 mm and a width W1 of about 0.02 mm. The port array 25 has an infusion length L2 of about 5.8 mm and provides a total infusion area of about 144.14 mm² and a combined total port area of about 0.192 mm².

As shown in FIG. 9B, an example port array 25 includes circular side-ports 124 arranged in a total of twelve (12) groups 100, each group 100 having three (3) ports 124. Each port 124 has a radius of about 0.05 mm. The groups 100 are arranged into three (3) rows 70 having four (4) groups per row 70 (i.e., a 3×4×3 array having a total of thirty-six (36) side-ports). This port array 25 provides an angular span of 360 degrees. Each row 70 has spacing angles A1 of about 90 degrees, and the middle row 70 is angularly offset at an angle A2 of about 45 degrees. The array 25 has an infusion length L2 of about 5.8 mm and provides a total infusion area of about 144.05 mm² and a combined total port area of about 0.283 mm².

As shown in FIG. 9C, an example port array 25 includes circular side-ports 124 arranged in thirty-one (31) rows 70 with four (4) ports per row 70 (i.e., a 31×4 array having a total of 124 side-ports) that provides an angular span of 360 degrees. Each port 124 has a radius of about 0.03 mm. Adjacent rows are spaced from each other at inter-row distances L8 of about 0.2 mm. Each row 70 has spacing angles A1 of about 90 degrees, and adjacent rows are angularly offset from each other at an angle A2 of about 45 degrees. The port array 25 has an infusion length L2 of about 6.06 mm and provides a total infusion area of about 143.98 mm² and a combined total port area of about 0.350 mm².

As shown in FIG. 9D, an example port array 25 includes circular side-ports 124 arranged in seven (7) rows 70 with four (4) ports per row 70 (i.e., a 7×4 array having a total of 28 side-ports) that provides an angular span of 360 degrees. Each port 124 has a radius of about 0.06 mm. Adjacent rows are spaced from each other at inter-row distances L8 of about 0.2 mm. Each row 70 has spacing angles A1 of about 90 degrees, and adjacent rows are angularly offset from each other at an angle A2 of about 45 degrees. The port array 25 has an infusion length L2 of about 6.12 mm and provides a total infusion area of about 144.01 mm² and a combined total port area of about 0.317 mm².

As shown in FIG. 9E, an example port array 25 includes circular side-ports 124 arranged in two (2) rows 70 with three (3) ports per row 70 (i.e., a 2×3 array having a total of six (6) side-ports) that provides an angular span of 360 degrees. Each port 124 has a radius of about 0.10 mm. Each row 70 has spacing angles A1 of about 120 degrees, and the rows are angularly aligned with each other. The port array 25 has an infusion length L2 of about 6.0 mm and provides a total infusion area of about 144.14 mm² and a combined port area of about 0.188 mm².

As shown in FIG. 9F, an example port array 25 includes elongated side-ports 24 arranged in fourteen (14) rows 70 with four (4) ports per row 70 (i.e., a 14×4 array having a total of fifty-six (56) side-ports) that provides an angular span of 360 degrees. Each row 70 has spacing angles A1 of about 90 degrees, and adjacent rows are angularly offset from each other at an angle A2 of about 45 degrees. Each side-port 24 has a length L9 of about 0.3 mm and a width W1 of about 0.02 mm. The port array 25 has an infusion length L2 of about 6.15 mm and provides a total infusion area of about 144.0 mm² and a combined port area of about 0.336 mm².

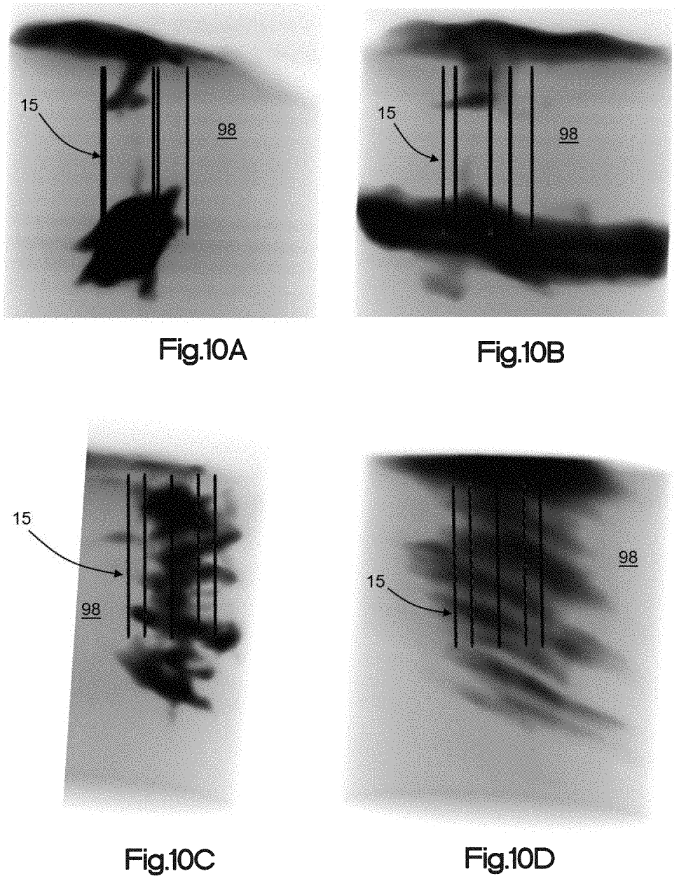

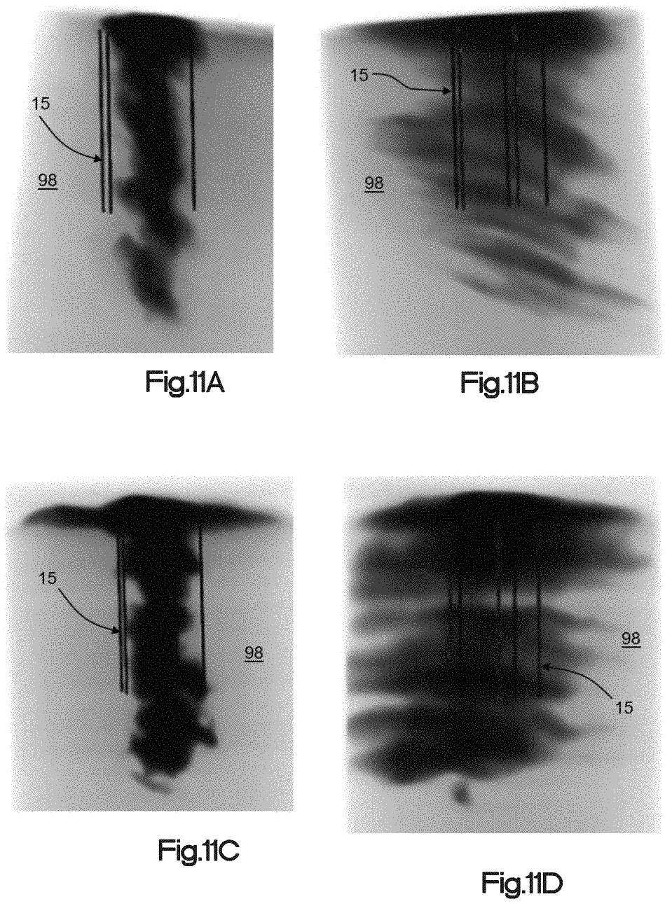

Referring now to FIGS. 10A-10D, in which test results show comparative fluid dispersions from a standard distal injection (i.e., bolus injection, shown in FIGS. 10A-10B) versus a side-port injection (FIGS. 10C-10D). Each injection employed an injectate volume of 1 mL into ex vivo pig muscle, observed by fluidic imaging. The fluidic images were reconstructed from high-resolution microCT scans, each taking from about 5-30 minutes to complete. For reference purposes, the electrode array 15 was superimposed in these fluidic images (specifically, the CELLECTRA 5P-IM Array, for exemplary purposes). It should be noted that the depicted array 15 employs a circular electrode pattern with a pattern diameter of 10 mm. Each pair of fluidic images (i.e., FIGS. 10A-10B and FIGS. 10C-10D) are taken at perpendicular views to each other, with FIG. 10A and FIG. 10C taken perpendicular to the direction of muscle fiber extension (i.e., the muscle fibers extend directly into and out of the page), and FIGS. 10B and 10D taken along the direction of muscle fiber extension (i.e, the muscle fibers extend left-to right). The bolus injection (FIGS. 10A-10B) was performed using a standard 21-guage (21G) injection needle having a single, distal opening at the end of the lumen. The side-port injection, shown in FIGS. 10C-10D, was performed with an injection needle 20 configured similarly to that shown in FIGS. 2A-2F, and specifically having a port array 25 that includes elongated side-ports 24 arranged in a 6×4 array spanning 360 degrees, with 90-degree spacing angles A1, and having the following additional port array parameters, as outlined in Table 1 below:

TABLE 1

| | | port dim | | | infusion | infusion | inj needle |
|---|---|---|---|---|---|---|---|
| | | (L9 × L1) | port SA | total SA | length L2 | depth L3 | depth L1 |
| n ports | port shape | (mm) | (mm²) | (mm²) | (mm) | (mm) | (mm) |
| 24 | rectangular | 0.8 × 0.02 | 0.016 | 0.384 | 13.3 | 3.6 | 22 |

It should be noted that, in Table 1, the term “port SA” refers to individual port surface area, and “total SA” refers to combined or total port surface area. The images demonstrate that the bolus injection (FIGS. 10A-10B) generally pooled around the distal tip of the standard injection needle, which pooling occurred in both visible planes (i.e., both along and perpendicular to muscle fiber extension). Moreover, the bolus injection dispersed such that a majority of the injectate remained located below the five (5) electrodes 14 of the electrode array 15. Comparatively, the side-port injection (FIGS. 10C-10D) dispersed in a more vertical columnar manner in both planes compared to the bolus injection, providing better vertical localization with respect to the five (5) electrodes 14 of the electrode array 15. The inventors observed that there are minimal diffusive changes in fluid dispersion during that time span (about 5-30 minutes) employed to generate the fluidic images shown in FIGS. 10A-10D. Moreover, because the subject tissue was ex vivo tissue, there were no convective changes (or at most only de minimis convective changes); therefore, these fluidic images should accurately represent the state of the injected fluid immediately following injection.

Referring now to FIGS. 11A-11D, additional test results show fluidic images that compare side-port fluid dispersions from a 1 mL injection (FIG. 11A-11B) and a 2 mL injection (FIGS. 11C-11D), using the same fluidic imaging techniques, superimposed electrode array, and port array parameters employed for the results shown in FIGS. 10C-10D. As above, both injections disperse favorably in a vertical columnar fashion. In the views taken along the direction of muscle fiber extension (FIGS. 11B and 11D), however, it appears that a greater proportion of the 2 mL injection is located outside the volume between the electrode array 15. This suggests that the 1 mL side-port injection may be more efficient at delivering the injectate between the electrodes. Referring now to FIG. 11E, a similar study was performed in rabbits using the same port array parameters (CELLECTRA 5P-IM Array), in side-port injections of 1 mL and 2 mL were followed by electroporation at equivalent EP parameters (1.0 Amp), to evaluate the effect of injection volume on dMAb expression. No fluidic or dMAb expression benefit was observed with the 2 mL injection, which suggests that 1 mL side-port injections are sufficient to take advantage of the benefits provided by rectangular side-ports of the present disclosure.

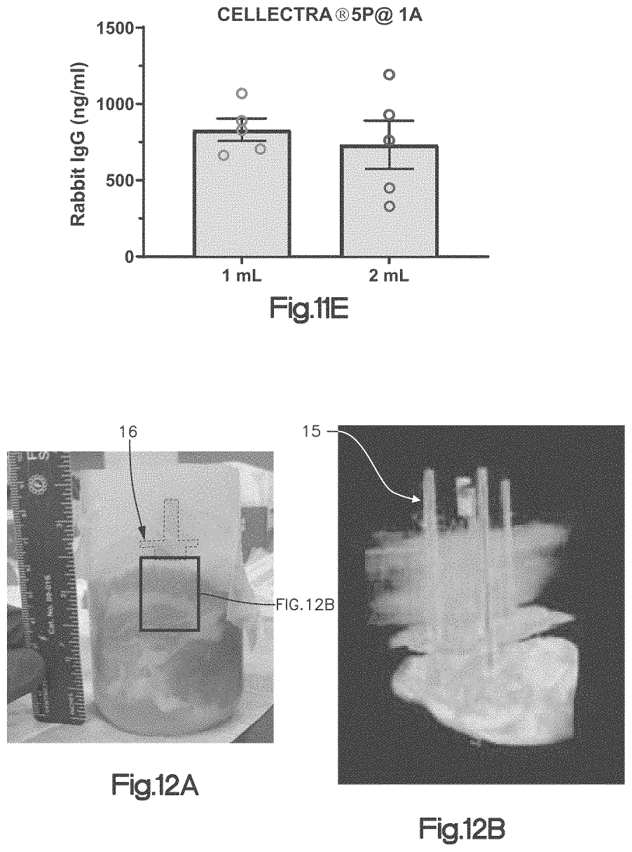

Referring now to FIGS. 12A-12B, additional test results show side-port fluid dispersion from the same injection needle 20 design employed in the tests shown in FIGS. 10C-10D. The injection was 2 mL into ex vivo pig muscle having dense intramuscular fat deposits. As shown in FIG. 12B, it can be seen that the fluid dispersed again in a vertically columnar manner, with favorably vertical localization with respect to the electrodes 14. It can also be seen that the injectate dispersion was able to overcome the intramuscular fat deposits, distributing injectate into all tissue contacting side-ports.

The side-port injection needles 20 described above are configured to enhance co-localization of the injectate with an electroporation field within target tissue, such as muscle tissue. The electroporation field is created by delivering one or more electroporative pulses of electrical energy through the electrode array 15 to the tissue. With reference to the hand-held electroporation device 4 described above (see FIGS. 1A-1D), the pulse generator 112 is configured to deliver an electroporation signal in the form of one or more electroporation pulses to the electrodes 14, which in turn deliver the one or more electroporation pulses to the tissue in contact with the electrodes 14, thereby creating an electroporation field within the tissue (e.g., intramuscular tissue). The electroporation field is tailored to substantially cause reversible poration in the cellular membranes of cells (e.g., muscle cells) within the field, causing transfection of the injectate (and agent(s) therein) into the temporarily porated cells. In this manner, the electroporation field can be said to create a transfection zone within the target tissue.

The one or more electroporation pulses delivered by the electrodes 14 can have an electric potential (voltage) in a range of about 5 V to about 1000 V (1 kV).

The one or more electroporation pulses can have an electric current (amperage) in a range of about 0.01 Amp to about 2.0 Amps and a pulse duration in a range of about 100 microseconds to about 500 milliseconds. The quantity of electroporation pulses can be in a range of 1 pulse to about 10 pulses, and more particularly in a range of about 3 pulses to about 5 pulses. For multi-pulse deliveries, each electroporation pulse can be separated in time from adjacent pulses by a pulse delay in a range of about 1 millisecond to about 1 second.

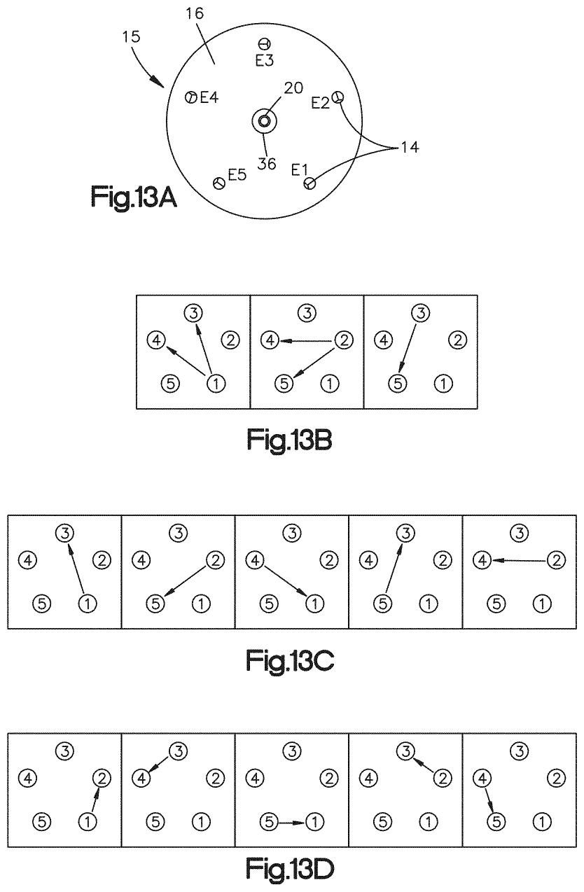

Referring now to FIG. 13A, the electrodes 14 of the electrode array 15 can be pulsed or “fired” according to one or more specific pulsing sequences, also referred to as “firing patterns.” As shown, the electrode array 15 according to the present example includes five (5) electrodes carried by the support member 16 and evenly spaced in a circular pattern. In this embodiment, the injection channel 36 of the support member 16 is preferably located at the center of the electrode array 15, thereby causing the injection needle 20 to be substantially equidistantly spaced from each electrode 14 in the array 15. For purposes of describing example pulse patterns employed by the electrode array 15, the electrodes 14 thereof can be referred to by electrode positions E1-E5. Referring now to FIG. 13B, a first example pulse pattern includes three (3) pulses, of which pulse 1 and pulse 2 are delivered from one active electrode to two return electrodes (thus splitting the current between two electrodes), and pulse 3 is delivered from one active electrode to one return electrode. For example, pulse 1 is delivered from E1 to E3 and E4; pulse 2 is delivered from E2 to E4 and E5; and pulse 3 is delivered from E3 to E5. Referring now to FIG. 13C, a second example pulse pattern, also referred to as a “star” pattern, includes five (5) pulses, each delivered from one active electrode to one return electrode, thus providing a more focused current path. For example, pulse 1 is delivered from E1 to E3; pulse 2 is delivered from E2 to E5; pulse 3 is delivered from E4 to E1; pulse 4 is delivered from E5 to E3; and pulse 5 is delivered from E2 to E4. Referring now to FIG. 13D, a third example pulse pattern, also referred to as a “perimeter” pattern, includes five (5) pulses, each delivered from one active electrode to one adjacent return electrode. For example, pulse 1 is delivered from E1 to E2; pulse 2 is delivered from E3 to E4; pulse 3 is delivered from E5 to E1; pulse 4 is delivered from E2 to E3; and pulse 5 is delivered from E4 to E5. It should be appreciated that the foregoing three pulse patterns represent non-limiting examples that can be employed with the electrode array 15. Some of these example pulse patterns were employed in the tests described below. Moreover, numerous other pulse patterns can be employed by the electrode array 15.

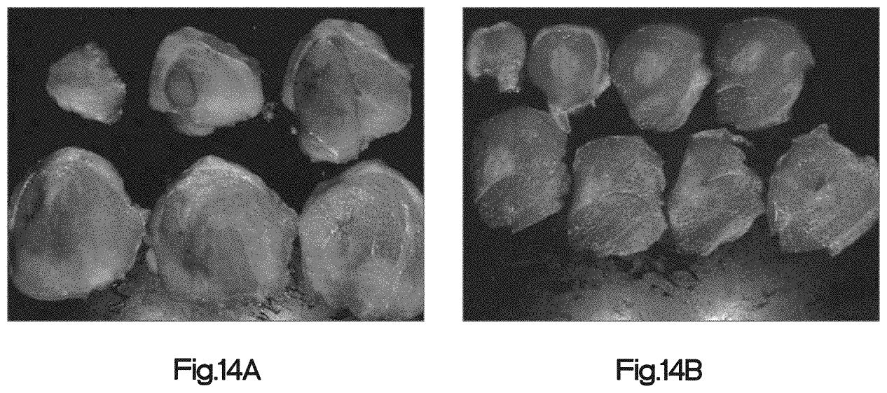

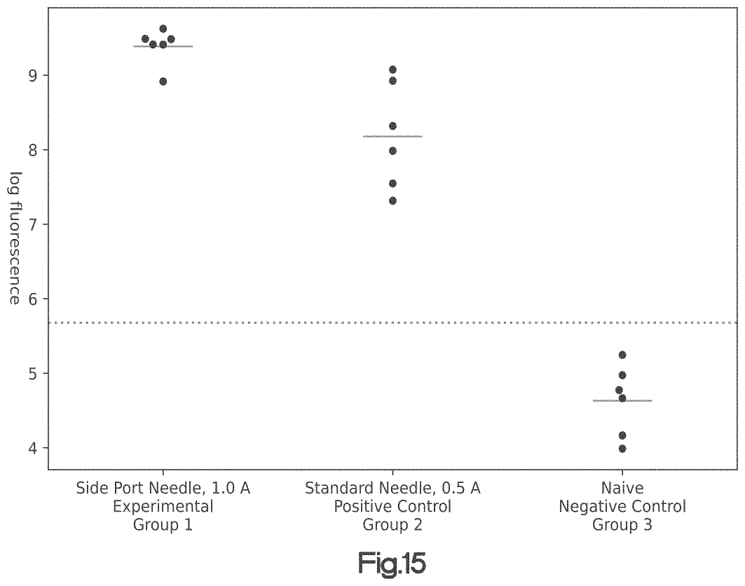

Referring now to FIGS. 14A-14B, a comparative study of the effect of side-port fluid injection and electroporation on cellular infiltration in muscle tissue was performed on rabbits. A plasmid encoding the gene for green fluorescent protein (GFP) was injected into the muscle tissue using the CELLECTRA® 5P-IM Applicator with a standard injection needle and with a side-port injection needle 20. For both groups, injection was followed with electroporation (EP) at the injection site using the CELLECTRA® 5P-IM Array at 1.0 Amp. Histological sections were taken at the treatment site at 3-days following the treatments for comparison of GFP expression (visible here as blue fluorescence) (nonspecific autofluorescence is visible here as green fluorescence). As shown, GFP expression (blue) is detectable in both the standard injection group (FIG. 14A) and the side-port injection group (FIG. 14B). Referring now to FIG. 15, in a similar study involving IM injection in rabbits, when a standard injection group was electroporated at 0.5 Amp, compared to 1.0 Amp for the side-port injection group, the GFP expression was about 10-times (10×) higher for the side-port injection/1.0 Amp group. Both groups demonstrated over 1000× increase in fluorescence over baseline (naïve) tissue. The side-port injection needle 20 used in this study employed twenty (20) rectangular ports 24 arranged in a 5×4 array spanning 360 degrees, with 90-degree spacing angles A1, and having the following additional port array parameters outlined in Table 2 below:

TABLE 2

| | | port dim | | | infusion | infusion | inj needle | |
|---|---|---|---|---|---|---|---|---|
| | | (L9 × W1) | port SA | total SA | length L2 | depth L3 | depth L1 | Inj |
| n ports | port shape | (mm) | (mm²) | (mm²) | (mm) | (mm) | (mm) | volume |
| 20 | rectangular | 0.8 × 0.04 | 0.032 | 0.64 | 10.8 | 6.1 | 22 | 1 mL |

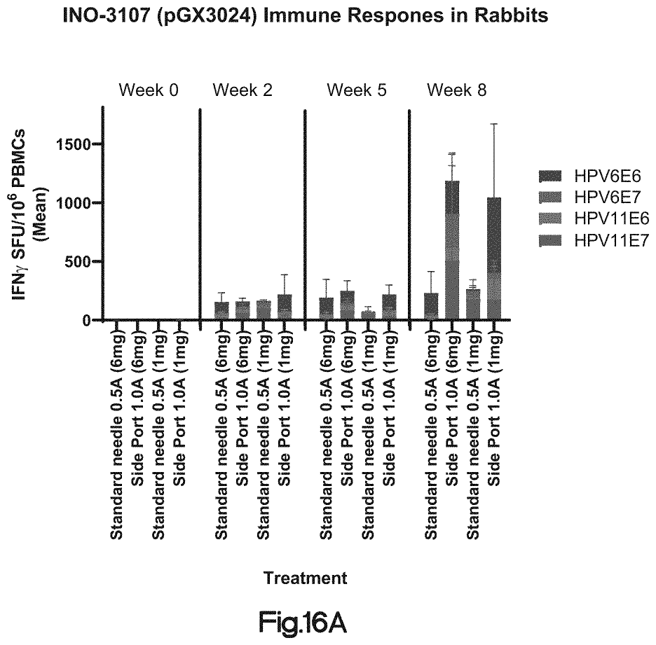

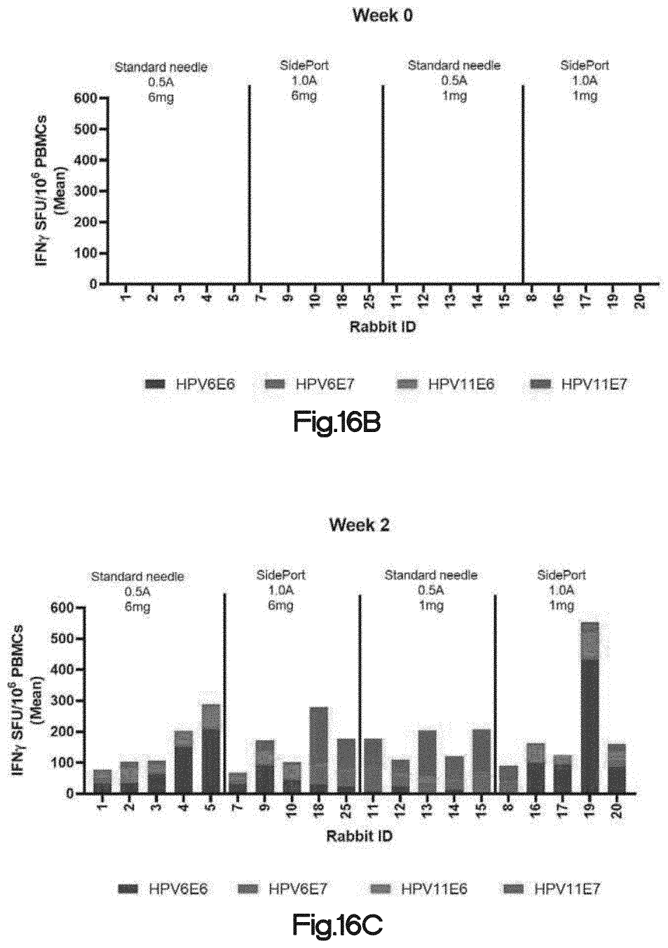

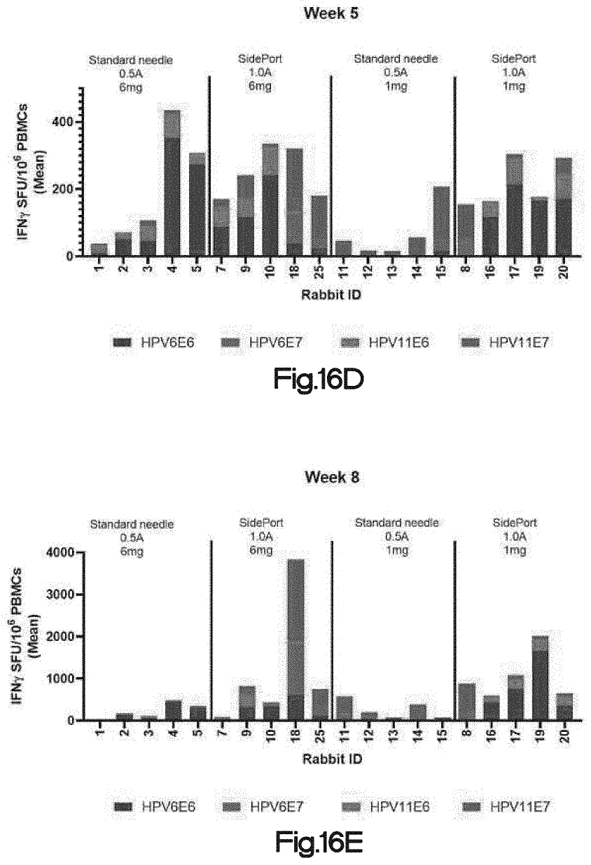

Referring now to FIGS. 16A-16E, the illustrated test results compare the level of immune responses induced by delivery of pGX3024 (a DNA plasmid encoding a human papillomavirus (HPV) antigen comprising SynCon® E6 and E7 antigens of both HPV6 and HPV11) with the CELLECTRA® 5P-IM Applicator with a standard injection needle (21G, single distal port) versus with a side-port injection needle 20, specifically the injection needle 20 described above with reference to FIG. 9A. The DNA plasmid pGX3024 was contained in immunologic composition INO-3107, which combines pGX3024 with pGX6010, the latter being a DNA plasmid encoding human vaccine adjuvant IL-12. For both injection types, injection was followed by electroporation (EP) using the needle array 15. The electroporation pulses were controlled by the CELLECTRA® 2000 electroporation system, employing the CELLECTRA® 5P-IM Array (needle array 15) coupled to the applicator head 10. The needle array 15 employed electrodes 14 having lengths L5 of 19 mm. Both the standard and side-port injection needles had a penetration depth L1 of 16 mm.

In this study, New Zealand White rabbits were randomized into groups of 5 and immunized three times at three-week intervals with INO-3107 formulated at 6 mg or 1 mg pGX3024 in 1 mL 1X SSC, by intramuscular (IM) injection with the standard injection needle versus the side-port injection needle 20. As described above, the side-ports allow for localization of the injectate within the electroporation field. Standard IM immunization (without side-port needle) by EP was performed at 0.5 ampere (Amp). Side-port needle IM immunization was performed with electroporation pulses at 1.0 Amp. Cellular immune responses were evaluated by IFNγ ELISpot before immunization (Week 0, shown in FIG. 16B) and two weeks after each immunization (Weeks 2, 5 and 8, shown in FIGS. 16C, 16D, and 16E, respectively). HPV6- and HPV11-specific T cell responses were detected in all rabbits following vaccinations. The use of IM injection coupled with the side-port needle demonstrated a dose-sparing response in IFNγ ELISpot data by week 5, and by week 8 the side-port delivery at either dose was superior to standard needle delivery at both doses. All groups demonstrated similar responses at week 2, while at week 5 the IM injection with the side-port needle at 1 mg dose and 1.0 Amp EP demonstrated similar responses to the 6 mg dose IM injection alone or with use of the side-port needle, suggesting a dose-sparing effect, and at week 8 both side-port groups had overall stronger responses than either standard IM delivery group (FIG. 16A). Overall, compared to standard IM delivery, delivery of vaccine by IM with side-port trended higher HPV responses by Week 8 in both dose groups but was not statistically significant (FIG. 16A). The data also shows that the immune responses were specific to both the E6 and E7 antigens for each of HPV6 and HPV11 following IM or IM with side-port needle delivery (FIG. 16B-16E).

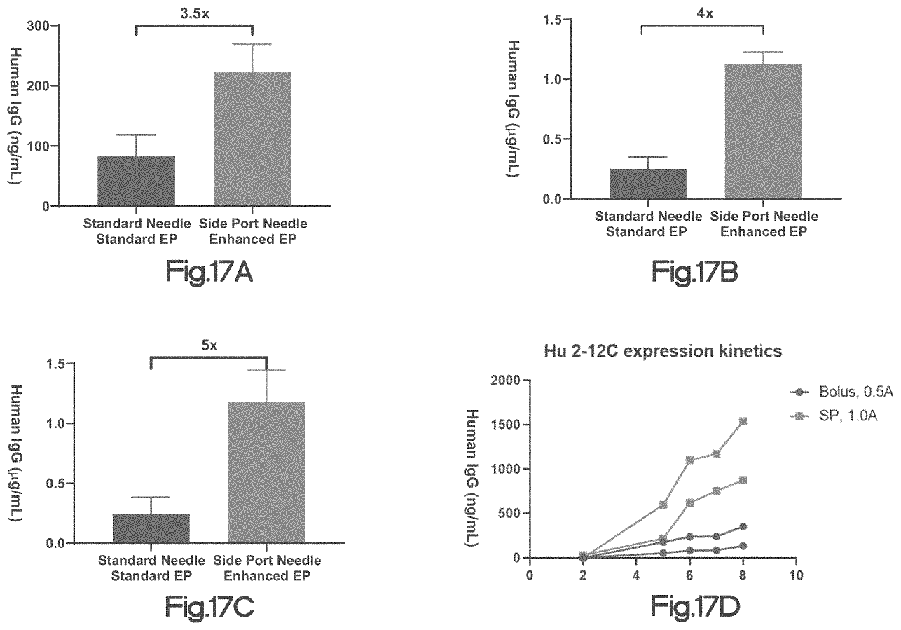

Referring now to FIGS. 17A-17D, a study compared dMAb expression in rabbits (FIG. 17A), rhesus monkeys (FIG. 17B), and pigs (FIG. 17C-17D), and following standard injection and electroporation at 0.5 Amp versus side-port injection and electroporation at 1.0 Amp. The side-port injection, 1.0 Amp EP groups demonstrated superior dMAb expression compared to the standard injection, 0.5 Amp EP groups across species (3.5× increase in rabbits, 5× increase in pigs, and 4× increase in rhesus monkeys.

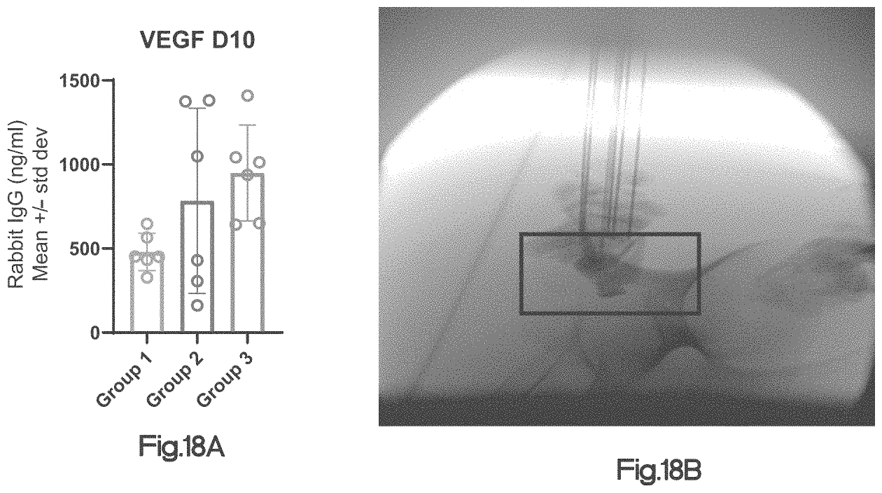

Referring now to FIG. 18A, the illustrated results compare the effect of side-port infusion length (L2) on dMAb expression in rabbits. Three (3) different side-port injection needles 20 were employed in respective Groups 1, 2 and 3, which needles having the port array parameters outlined in Table 3 below:

TABLE 3

| | | | port dim | | | infusion | infusion | inj needle |
|---|---|---|---|---|---|---|---|---|
| | | | (L9 × W1) | port SA | total SA | length L2 | depth L3 | depth L1 |
| Group | n ports | port shape | (mm) | (mm²) | (mm²) | (mm) | (mm) | (mm) |
| 1 | 20 | rectangular | 0.8 × 0.02 | 0.016 | 0.320 | 10.8 | 6.1 | 22 |
| 2 | 16 | rectangular | 0.8 × 0.02 | 0.016 | 0.256 | 8.3 | 5.6 | 19 |
| 3 | 12 | rectangular | 0.8 × 0.02 | 0.016 | 0.192 | 5.8 | 5.1 | 16 |

This study demonstrates that reducing the infusion length L2 from 10.8 mm to 5.8 mm while concomitantly decreasing the injection needle depth L1 from 22 mm to 16 mm increased dMAb expression in rabbits. It is believed the increase results from less fluid dispersion/leakage out of the target area (and away from the intended electroporation field). In early designs of side-port injection needles 20, the distal end of the infusion zone was aligned with the distal ends 19 of the electrodes 14. However, the present study demonstrates benefits with providing a shorter infusion length and a proximal offset L6 (see FIG. 1E) from the distal ends 19 of the electrodes 14 and the distal end of the infusion zone. These results are further supported by imagery, such as that shown in FIG. 18B, in which the side-port fluid dispersion can be seen pooling distally below the electrode ends 19 and out of the electroporation zone. It should be noted that the distal end of the infusion zone employed in FIG. 18B was aligned with the distal ends 19 of the electrodes 14, demonstrating that a proximal offset L6 is preferable.

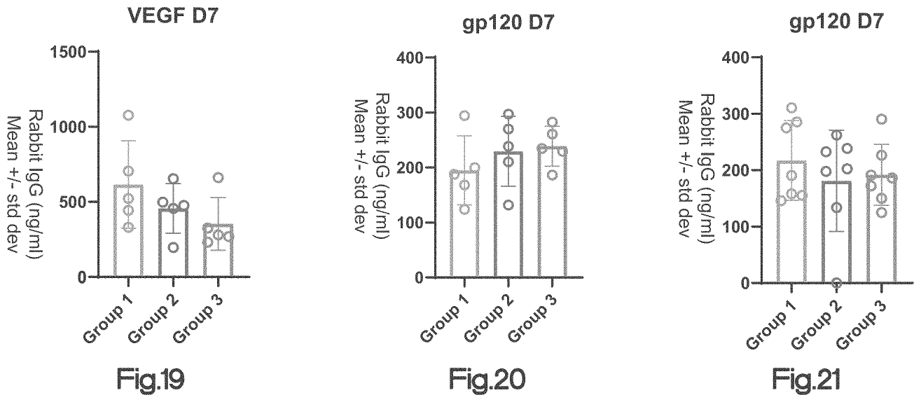

Referring now to FIG. 19, the illustrated results compare the effect of side-port shape on dMAb expression in rabbits. Three (3) different side-port injection needles 20 were employed in respective Groups 1, 2 and 3 according to the port array parameters outlined in Table 4 below:

TABLE 4

| | | | | | | infusion | infusion | inj needle |
|---|---|---|---|---|---|---|---|---|
| | | | | port SA | total SA | length L2 | depth L3 | depth L1 |
| Group | n ports | port shape | port dim | (mm²) | (mm²) | (mm) | (mm) | (mm) |
| 1 | 12 | rectangular | 0.8 × 0.02 | 0.016 | 0.192 | 5.8 | 5.1 | 16 |
| 2 | 36 | circular (arranged | 0.05 (radius) | 0.0079 | 0.283 | 5.8 | 5.1 | 16 |
| | | with 3x circles per | | | | | | |
| | | rectangular port | | | | | | |
| | | from group 1 | | | | | | |
| 3 | 28 | circular | 0.06 (radius) | 0.0113 | 0.317 | 6.1 | 5.1 | 16 |

In this study, the inventors found, surprisingly and unexpectedly, that the array of twelve (12) elongated rectangular side-ports in a 4×3 array (Group 1, see also FIG. 9A), demonstrated increased dMAb expression over an array of thirty-six (36) circular side-ports grouped together in triads in a manner generally approximating the rectangular side-ports (Group 2, see also FIG. 6), and demonstrated even further increased dMAb expression over an array of twenty-eight (28) homogenous circular ports of 0.06 mm radius (Group 3, see also FIG. 9D). The inventors previously believed that side-port arrays comprising smaller circular ports in increased numbers provided enhanced transfection. Thus, it was unexpected that fewer, elongated rectangular side-ports would result in increased transfection. Having performed numerous additional tests and studies, though desiring not to be bound by any particular theory, the inventors believe that one reason the rectangular side-ports provide increased expression is because they can span multiple muscle fibers, effectively allowing the fluid exiting the side-ports to take the path of least resistance along various muscle fibers. In this way, the inventors believe that rectangular ports with their long axis oriented to span muscle fibers is a more efficient use of port surface area than other port shapes such as circular ports.

Referring now to FIG. 20, the illustrated results compare the effect of side-port shape on dMAb expression in rabbits, while further maintaining the total side-port area substantially constant. Three (3) different side-port injection needles 20 were employed in respective Groups 1, 2 and 3 according to the port array parameters outlined in Table 5 below:

TABLE 5

| | | | | | | infusion | infusion | inj needle |
|---|---|---|---|---|---|---|---|---|
| | | | port dim | port SA | total SA | length L2 | depth L3 | depth L1 |
| Group | n ports | port shape | (mm) | (mm²) | (mm²) | (mm) | (mm) | (mm) |
| 1 | 124 | circular | 0.03 (radius) | 0.0028 | 0.350 | 6.1 | 5.1 | 16 |
| 2 | 28 | circular | 0.06 (radius) | 0.0113 | 0.317 | 6.1 | 5.1 | 16 |
| 3 | 56 | Rectangular | 0.3 × 0.02 | 0.006 | 0.336 | 6.2 | 5.1 | 16 |
| | | (short) | | | | | | |

The results demonstrate that small rectangular side-ports (Group 3) and outperformed larger circular ports (Group 2), and both outperformed the smaller circular ports (Group 1). As before, the inventors found these results surprising, as they challenged the previous notion that greater numbers of smaller circular ports would provide a more uniform fluid dispersion. It should further be noted that the larger rectangular side-ports from the study shown in FIG. 19 provide a larger magnitude improvement (Group 1 vs Group 3, FIG. 19) than the small rectangular side ports using the same comparator group (Group 3 vs Group 2, FIG. 20), which suggests that the larger rectangular side-ports from the study shown in FIG. 19 (Group 1) is most preferred of all designs tested.

Referring now to FIG. 21, the illustrated results compare the effect of injection rate through rectangular side-ports on dMAb expression in rabbits. In this study, Group 1 (medium rate) was injected at a rate of 5 seconds per mL. Group 2 (slow injection) was injected at 30 seconds per mL. Group 3 (fast injection) was injected at a rate of 1 second per mL. Each of the Groups in this study used equivalent arrays of rectangular side-ports according to the port array parameters outlined in Table 6 below:

TABLE 6

| | | | port dim | | | infusion | infusion | inj needle | | |
|---|---|---|---|---|---|---|---|---|---|---|
| | | | (L9 × W1) | port SA | total SA | length | depth | depth L1 | inj rate | |
| Group | n ports | port shape | (mm) | (mm²) | (mm²) | L2 (mm) | L3 (mm) | (mm) | (s/mL) | |
| 1 | 12 | rectangular | 0.8 × 0.02 | 0.016 | 0.192 | 5.8 | 5.1 | 16 | 5 | s |
| 2 | 12 | rectangular | 0.8 × 0.02 | 0.016 | 0.192 | 5.8 | 5.1 | 16 | 30 | s |
| 3 | 12 | rectangular | 0.8 × 0.02 | 0.016 | 0.192 | 5.8 | 5.1 | 16 | <1 | s |

This study demonstrates that injection rate through these rectangular side-ports did not have a significant impact on dMAb expression.

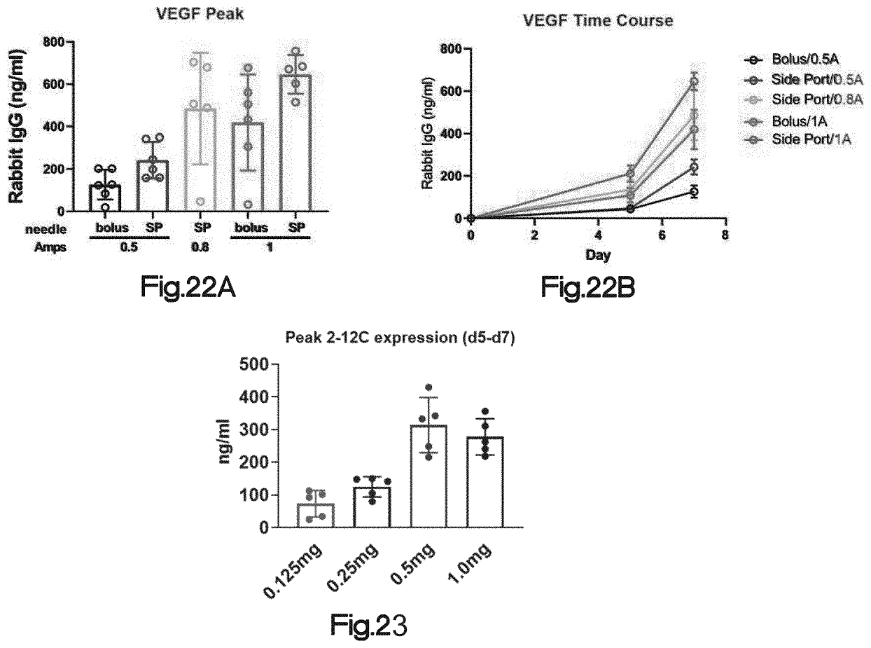

The inventors have performed additional studies to identify and evaluate beneficial electroporation parameters for use with the side-port arrays described herein. Unless stated otherwise, the studies described with reference to FIGS. 22-29 utilized the pulsing pattern described above with reference to FIG. 13B.

Referring now to FIG. 22, this study was designed to identify the interaction between the injection method (side port vs standard needle) and the electroporation amperage (0.5, 0.8, or 1.0 Amp pulse current with a 200 Volt maximum pulse voltage). Side-port delivery was shown to enhance dMAb expression compared to standard needle delivery independent of electroporation parameters, and similarly, increasing electroporation amperage enhanced dMAb expression for both side-port and standard needle delivery. The combination of 1.0 Amp pulse current and side-port delivery provided the highest and most consistent dMAb expression in this study.

Referring now to FIG. 23, this study was designed to evaluate the impact of plasmid concentration on side-port delivery in rabbits. Plasmid concentrations below 0.25 mg significantly reduced dMAb expression, while both 0.5 mg/mL and 1.0 mg/mL concentrations resulted in comparable expression levels.

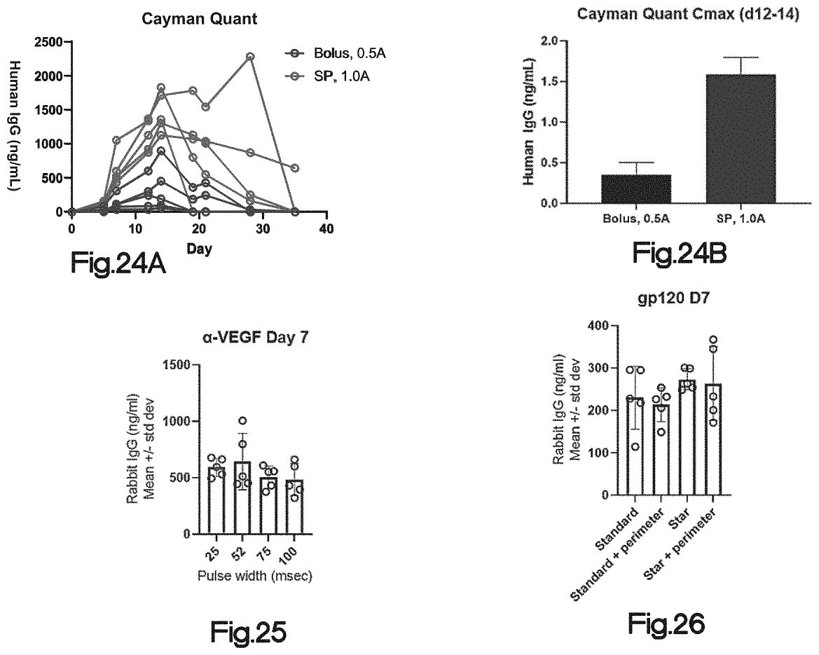

Referring now to FIGS. 24A-24B, this study was designed to compare two (2) dMAb delivery methods in nonhuman primates. Side-port delivery with 1.0 Amp EP amperage generated superior dMAb expression compared to standard needle delivery with 0.5 Amp EP amperage. This study, in combination with the studies shown in FIGS. 17A-17D, demonstrate that a delivery regime utilizing side-port injection with EP at 1.0 Amp is generally superior to a delivery regime utilizing standard injection with EP at 0.5 Amp.

Referring now to FIG. 25, this study was designed to evaluate the impact of pulse duration on dMAb expression following side-port delivery in rabbits. Pulse widths of 25 msec and 52 msec provided comparable dMAb expression, and increasing pulse width to 75 msec or 100 msec provided progressively lower expression levels, suggesting that increasing pulse duration beyond 52 msec is potentially detrimental when using this device configuration.

Referring now to FIG. 26, this study was designed to evaluate the impact of different pulse firing patterns on dMAb expression following side-port delivery in rabbits, particularly the “star” pulse pattern (FIG. 13C) and “perimeter” pulse pattern (FIG. 13D) described above. The “star” pulse pattern generated the highest mean expression levels with the lowest variability, suggesting that this pulse pattern may be beneficial for dMAb delivery with this device configuration. This study suggests that the addition of this “perimeter” pulse pattern was not beneficial to dMAb expression.

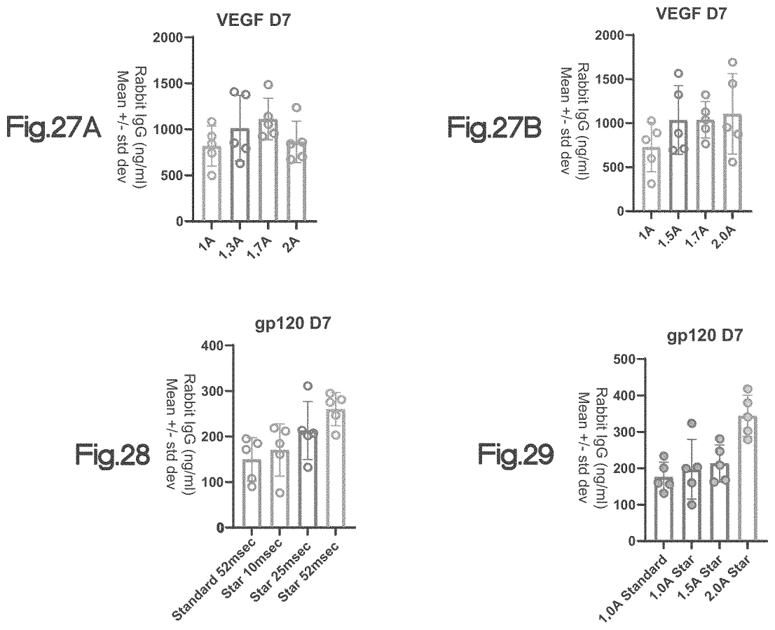

Referring now to FIG. 27A, this study was designed to evaluate the impact of pulse amperages above 1.0 Amp on dMAb expression following side-port delivery. Increasing pulse amperage from 1.0 Amp up to 1.7 Amps increased dMAb expression, while further increasing pulse amperage to 2.0 Amps reduced dMAb expression, suggesting that an amperage in the range of 1.3 Amps to 1.7 Amps may be preferred over the other tested amperages for this particular device configuration. In a follow-up study, shown in FIG. 27B, a similar range of pulse amperages were evaluated in rabbits and again, 1.7 Amps was found to be a preferred current of those tested.

Referring now to FIG. 28, this study was designed to evaluate the impact of pulse duration on the “star” pulse pattern (FIG. 13C) following dMAb delivery in rabbits. Here, the “star” pattern used a pulse amperage of 2.0 Amps while the “standard” pulse pattern (FIG. 13B) used a pulse amperage of 1.0 Amp. Increasing pulse duration of the “star” pulse pattern from 10 msec to 25 msec to 52 msec progressively increased dMAb expression, suggesting that pulse durations below 52 msec may be detrimental to dMAb expression with this pulse pattern.

Referring now to FIG. 29, this study was designed to evaluate the impact of pulse amperage on the “star” pulse pattern (FIG. 13C) following side-port dMAb delivery in rabbits. As a control, the “standard” pulse pattern (FIG. 13B) at 1.0 Amp was used. Increasing pulse amperage from 1.0 Amp to 1.5 Amps had no measurable effect on dMAb expression, while increasing the amperage to 2.0 Amp provided a substantial increase in dMAb expression. The inventors found these results surprising in view of the results shown in FIGS. 27A-27B, which indicated that dMAb is better expressed at 1.7 Amp using the standard pulse pattern. The results shown in FIG. 29 suggest that different pulse patterns may have different preferred pulse amperages.

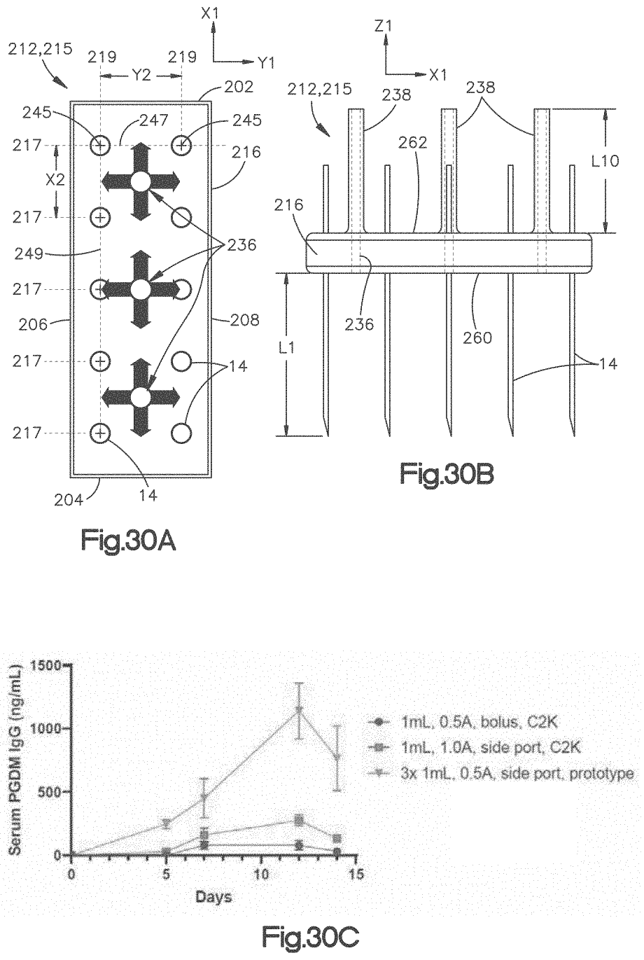

Referring now to FIGS. 30A-30B, FIGS. 31A-32B, and FIG. 33, various embodiments of array assemblies 212 can employ multiple injection channels for respective multiple side-port injection needles 20. Such array assemblies 212 can be configured to provide increased injection volumes, particularly with enhanced co-localization with larger electroporation fields.

As shown in FIGS. 30A-30B, an example array assembly 212 includes a support member 216 carrying an array 215 of needle electrodes 14 arranged in a grid or “matrix” pattern. The illustrated embodiment employs a matrix having five (5) rows 217 and two (2) columns 219 of electrodes 14 (i.e., a 5×2 electrode array 215, in which each row has two electrodes, and each column has five electrodes). The rows 217 are spaced at intervals along a longitudinal direction X1, while the columns 219 are spaced at intervals along a lateral direction Y1 that is substantially perpendicular to the longitudinal direction X1. In this manner, the array 215 can be elongated along the longitudinal direction X1. It should be appreciated that the electrodes 14 of each row 217 can be aligned along a row axis 247, which can intersect central axes 245 of the electrodes 14 in the row 217. Additionally, the electrodes 14 of each column 219 can be aligned along a column axis 249, which can intersect the central axes 245 of the electrodes 14 in the row 219. The array 215 can employ equidistant row and column spacing X2, Y2, although in other embodiments the row spacing X2 can differ from the column spacing Y2. The row and column spacing X2, Y2 is preferably measured between adjacent row axes 247 and column axes 249, respectively. The electrodes 14 can be configured similarly to those described above with respect to the circular pattern electrode arrays 15, although in other embodiments the electrodes 14 of the present array 215 can be adapted as needed.

The support member 216 has first and second ends 202, 204 opposite each other along the longitudinal direction X1 and opposed first and second sides 206, 208 opposite each other along the lateral direction Y1. A bottom surface 260 of the support member can effectively define a stop surface that is configured to contact the patient's skin and control the depths at which the electrodes 14 penetrate the tissue. The support member 216 preferably includes a plurality of injection channels 236 extending through the array 215. As shown, the support member 216 can include three (3) injection channels 236, which can be aligned with each other along the longitudinal direction X1 and can be equidistantly spaced between the first and second columns 219. A first one of the injection channels 236 can also be equidistantly positioned between the first and second rows 217, a second one of the injection channels 236 can be laterally aligned in the third row 217, and a third one of the injection channels 236 can be equidistantly positioned between the fifth and sixth rows 217. One or more and up to all of the injection channels 236 can be defined within vertically elongated chimneys 238. Each chimney 238 can be configured to receive a respective side-port injection needle 20, which can be configured according to any of the embodiments described above. As shown in FIG. 30B, the electrodes 14 can extend distally from the support member 216 to an electrode depth L1, and the chimneys 238 can extend from an upper surface 262 of the support member 216 proximally to a chimney height of L10 along a vertical direction Z1, which can be configured to place the infusion regions of the injection needles 20 at a favorable position relative to distal ends 19 of the electrodes 14, such as at a favorable electrode offset distance L6 described above.

As shown in FIG. 30A, the side-port injections can each disperse their injectate radially outward toward the adjacent needle electrodes 14. In this manner, the array 215 can be configured to disperse greater volumes of injectate within larger electroporation fields. According to one example of the present embodiment, the array 215 can be configured to deliver a total injection volume of about 3 mL from the injection needles 20, particularly at 1 mL per injection needle 20. It should be appreciated that, when used for intramuscular (IM) electroporation, the elongated array 215 allows a physician to orient the array 215 so that that the longitudinal direction X1 generally aligns with the direction of muscle fiber extension, thereby further enhancing the fluid dispersion in the muscle tissue of the patient. It should be appreciated that, in most instances in intramuscular tissue, the natural flow or dispersion of the injected fluid and the bulk of the injectate is along the direction of the muscle fibers. One reason for this is because the lowest impedance to the flow of the fluid is in the longitudinal direction of the fibers. Thus, a physician can elect to apply the electroporation field in a perpendicular direction across the muscle fibers and injected fluid, such that more of the myocyte cells (i.e., muscle cells) can be transfected, taking advantage of this natural fluid distribution.

Referring now to FIG. 30C, the increased-volume array 215 described above was tested to evaluate dMAb expression in rabbits following increased volume (3 mL) side-port IM injection compared to the circular array 15 described above at 1 mL using both standard injection needles and side-portion injection needles 20. In particular, the array 215 used three (3) side-port injection needles 20, each injecting 1 mL into muscle, followed by the array 215 delivering EP at 0.5 Amp (triangle data markers). For comparison, the circular array 15 was the CELLECTRA® 5P-IM Array, using a standard injection of 1 mL, followed by EP at 0.5 Amp (circle data markers), and using a side-port injection of 1 mL, followed by EP at 1.0 Amp injection (square data markers). As shown, the increased-volume array 215 provided substantially immediate higher dMAb expression over the other groups, which further increased over the other groups, and remained higher over the course of the study (14 days). This study suggests that the increased-volume array 215 can provide significant enhancements in gene expression, even at a lower amperage.

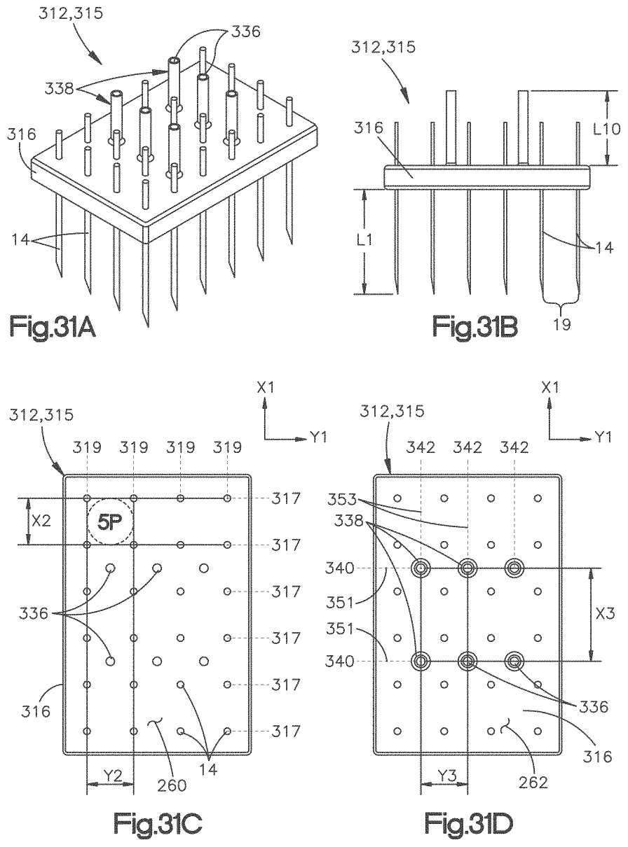

Referring now to FIGS. 31A-31D, another example array assembly 312 includes a support member 316 having an array 315 of needle electrodes 14 arranged in a matrix having six (6) rows 317 and four (4) columns 319 (i.e., a 6×4 matrix electrode array 315). As above, the rows 317 are spaced at intervals along the longitudinal direction X1, while the columns 319 are spaced at intervals along the lateral direction Y1, such that the array 315 can be elongated along the longitudinal direction X1. The array 315 can employ equidistant row and column spacing. By way of a non-limiting example, the rows 317 can be spaced from each other at a distance X2 of about 10 mm and the columns 319 can be spaced from each other at a distance Y2 of about 10 mm. It should be appreciated that such 10 mm spacing approximates the diameter of the circular electrode array of the CELLECTRA® 5P-IM Array, as shown for reference in FIG. 31C.

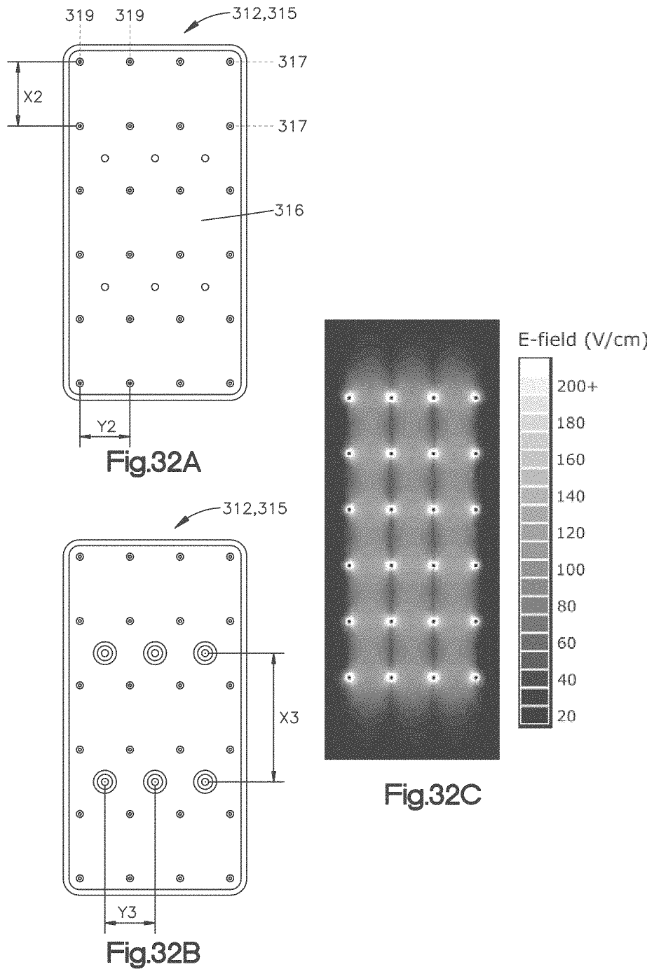

In other embodiments, as shown in FIGS. 32A-32B, the row spacing can differ from the column spacing. In this example, the columns can be spaced at distances X2 of about 10 mm, and the rows can be spaced at distances Y2 of about 7.5 mm. Additional spacing distances are discussed below.

The support members 316 of the arrays 315 shown in FIGS. 31A-32B preferably includes a plurality of injection channels 336, which can be defined within vertically elongated chimneys 338. As shown, the plurality of injection channels 336 can include six (6) injection channels 336, which can be arranged along two (2) rows 340 of channels, such as a first row 340 of channels 336 equidistantly spaced between the second and third rows 319 of electrodes 14, and a second row 340 of channels 336 equidistantly spaced between the fourth and fifth rows 319 of electrodes 14. As shown in FIG. 31D, the channel rows 340 can be spaced from each other at spacing distance X3, as measured between respective channel row axes 351 that intersect central axes 355 of the injection channels 336 in the channel row 340. In the illustrated embodiment, spacing distance X3 is 2× the electrode row spacing distance X2. The channels 336 can also be arranged into columns 342 of channels 336, such as a first, second, and third column 342 of channels 336. The channel columns 342 can be spaced from each other at spacing distance Y3, as measured between respective channel column axes 353 that intersect the central axes 355 of the injection channels 336 in the channel column 342. In the illustrated embodiment, spacing distance Y3 is equivalent to the electrode column 319 spacing distance.

According to one example of the present embodiments, the arrays 315 can be configured to deliver a total injection volume of about 6 mL from the injection needles 20, particularly at 1 mL per injection needle 20. It should be appreciated that the arrays 315 can be used for delivering injection volumes greater than 6 mL and less than 6 mL. As with the array 215 described above, the present arrays 315 can be oriented favorably with respect to the direction of muscle fiber extension, thereby enhancing the fluid dispersion in the muscle tissue. Additionally, the chimneys 338 have heights L10 that can be configured to place the infusion regions of the injection needles 20 at a favorable position relative to distal ends 19 of the electrodes 14. It should be appreciated that the electrode and channel spacing distances X2, Y2, X3, Y3, electrode depths L1, and/or the chimney heights L10 of the matrix arrays 215, 315 described above can be varied as needed. For example, spacing distances X2, Y2, X3, Y3 can be in a range from about 2.5 mm to about 50 mm, and more particularly in a range from about 4.0 mm to about 20 mm, and more particularly in a range from about 5.0 mm to about 15.0 mm. The electrode spacing distances X2, Y2 along the direction of muscle fiber extension is preferably in a range of about 10.0 mm to about 15.0 mm. The electrode spacing distances X2, Y2 along a directional that is perpendicular to the direction of muscle fiber extension is preferably in a range of about 5.0 mm to about 10.0 mm. It should be appreciated that the foregoing spacing distances can be particular to the anatomy of the target tissue, particularly when the target tissue has anisotropic electrical and fluidic properties.

Referring now to FIG. 32C, a computer model illustrates an example of an electric field generated by the array 315 shown in FIGS. 32A-32B. As shown, the electric field can have a substantially even field magnitude, shown in V/cm, along the longitudinal direction X1 between adjacent columns. In this manner, the array 315 can provide both favorable longitudinal fluid dispersion (particularly when aligned with the muscle fibers), and favorable “smooth” electroporation fields along the longitudinal direction X1.

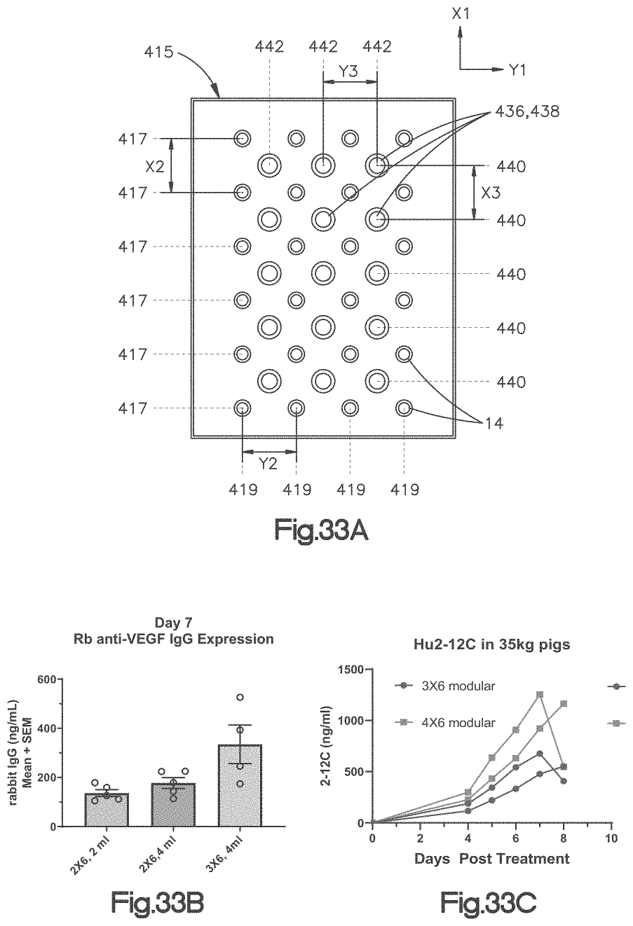

In further embodiments, the matrix arrays 215, 315 can be further configured for selective or “modular” use of the electrodes 14 and/or injection channels 236, 336 thereof. Referring now to FIG. 33A, an example array 415 having electrodes 14 arranged in a matrix, such as a 6×4 matrix with even electrode row 417 and column 419 spacing X2, Y2, by way of a non-limiting example, can include a total of fifteen (15) chimneys 438 (and channels 436), arranged in rows 440 and columns 442 in a 5×3 chimney array configured such that each chimney 438 is equidistantly spaced between the adjacent columns 419 and rows 417 of the electrodes 14. The array 415 can include circuitry for connecting each electrode 14 individually to the pulse generator 112, such that the pulse generator 112 can deliver electroporation pulses to any subset of the electrodes 14. Similarly, any subset of the chimneys 438 can be employed to receive a respective side-port injection needle 20. In this manner, a single matrix array 438 can provide the functionality of numerous matrix arrays 438. For example, the depicted 6×4 matrix array can be selectively employed as any of a 1×1, 1×2, 1×3, 1×4, 2×1, 2×2, 2×3, 2×4, 3×1, 3×2, 3×3, 3×4, 4×1, 4×2, 4×3, 4×4, 5×1, 5×2, 5×3, 5×4, 6×1, 6×2, 6×3, and 6×4 electrode array, utilizing any one of a 1×1, 1×2, 1×3, 2×1, 2×2, 2×3, 3×1, 3×2, 3×3, 4×1, 4×2, 4×3, 5×1, 5×2, and 5×3 chimney array.

Referring now to FIG. 33B, the 6×4 modular array 415 was tested to evaluate gene expression in two (2) Groups of pigs. Group 1 (circle data markers) was injected with 4 mL via side-port injection at chimney rows 2 and 4 (using only chimney columns 1 and 2) (1 mL side-port injection per chimney) and electroporated with associated 5×3 array subset. Group 2 (square data markers) was injected with 6 mL via side-port injection at chimney rows 2 and 4 (1 mL side-port injection per chimney) and electroporated with the entire 6×4 array. Group 2 demonstrated increased gene expression, suggesting that dispersing greater injection volumes along a more voluminous EP field can increase gene expression.

Referring now to FIG. 33C, a similar modular array study compared gene expression in three (3) Groups of rabbits. Group 1 was injected with 2 mL via side-port injection at chimney rows 2 and 4 (using only chimney column 1) (1 mL side-port injection per chimney) and electroporated with the associated 6×2 array subset. Group 2 was injected with 4 mL via side-port injection at three (3) chimney rows in chimney column 1 (1 mL side-port injection at chimney rows 2 and 4, and 2 mL side-port injection at chimney row 3) and electroporated with the associated 6×2 array. Group 3 was injected with 4 mL via side-port injection at chimney rows 2 and 4, chimney columns 1 and 2 (1 mL side-port injection per chimney) and electroporated with the associated 6×3 array. As shown at day 7 following treatment, Group 3 demonstrated nearly 2× the gene expression as Group 2, although Group 2 only moderately outperformed Group 1. This study further demonstrates that increased injection volume enhances gene expression when it is spatially dispersed to cover more tissue volume and appropriately paired with a more voluminous EP field.

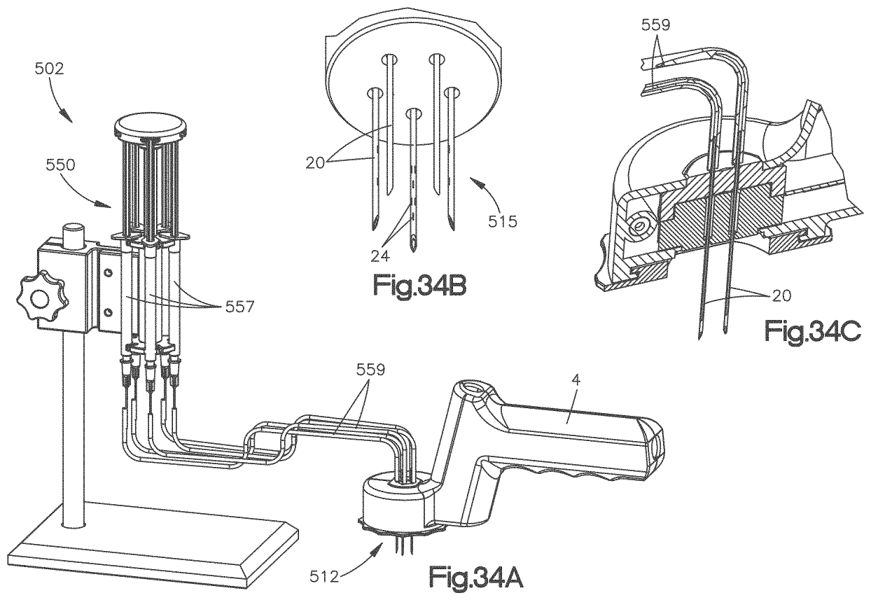

It should be appreciated that the electrode arrays 15, 215, 315, 415 described above can be adapted such that one or more and up to all of the needle electrodes is also a side-port injection needle, which needle can perform both fluid delivery and electroporation pulse delivery. Such dual-purpose needles can be referred to as “injection needle electrodes.” For example, referring now to FIGS. 34A-34C, an electroporation system 502 can include a hand-held electroporation device 4 that employs an electrode array assembly 512 that is configured similar to those shown in FIGS. 1A-1E, yet further adapted such that each of the needle electrodes is a dual-purpose side-port injection needle electrode 525. Each of the injection needle electrodes 525 in this example embodiment is in electrical communication with the pulse generator 112 and is also in fluid communication with a reservoir of injectate. In this example embodiment, the array assembly 512 is connected to a fluid delivery system 550 that carries a respective syringe 557 for each side-port injection needle electrode 525 and is configured to actuate each syringe 557 to inject a volume of the injectate into the target tissue. In such embodiments, each syringe 557 can carry one-fifth (⅕) of the total drug volume to be delivered. It should be appreciated that in such embodiments, the side-port injection needle electrodes 525 can cooperatively and effectively force a distribution of an injectate into a location intermediate the array of injection needle electrodes 525, and thereby need not rely upon the injectate to diffuse within tissue into the desired location.

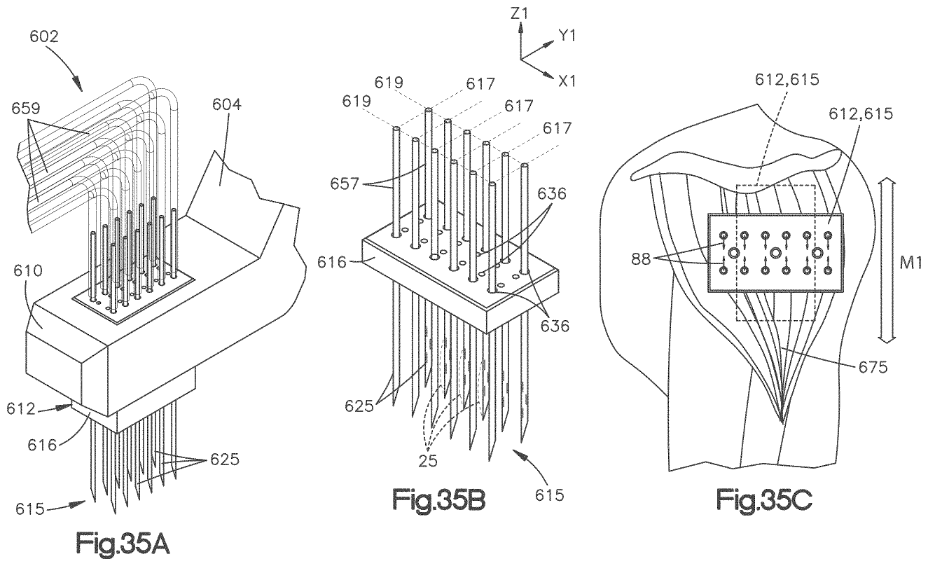

Referring now to FIGS. 35A-35C, an example of an electroporation system 602 is shown that includes an electrode array assembly 612 having a plurality of needle electrodes 625 arranged in rows 617 and columns 619 in a matrix array 615, generally similar to the embodiments described above with reference to FIGS. 30A-30B, FIGS. 31A-32B, and FIG. 33. However, in the present embodiment, one or more and up to all of the needle electrodes 625 in the matrix array 615 can be a dual-purpose injection needle electrode 625 that is configured to both inject fluid within target tissue and also to deliver one or more electroporative pulses to the target tissue.

As shown in FIG. 35A, the electroporation system 602 of this embodiment can include tubing 659 for delivering the fluid injectate to each dual-purpose injection needle electrode 625 in the matrix array 615. The tubing 659 can connect proximal ends 657 of the dual-purpose injection needle electrodes 625 to a reservoir, such as via a manifold of a reservoir assembly and/or via a plurality of individual reservoirs, which can be similar to those of the fluid delivery system 550 shown in FIG. 34A. The array assembly 612 can be configured to couple with an applicator head 610 of a hand-held electroporation device 604. For example, the array assembly 612 can include a support member 616 configured to couple with one or more complimentary mounting members of the applicator head 610, similar to the manner described above with reference to FIG. 1B. The dual-purpose electrodes 625 can extend through dual-purpose channels 636 defined through the support member 616. It should be appreciated that the support member 616 can be employed in modular fashion, similar to the manner described above with reference to FIG. 4. For example, the dual-purpose electrodes 625 can be inserted within a select sub-set of the available dual-purpose channels 636, which sub-set can be selected based on the fluid delivery and electroporation field parameters needed, which parameters (and thus sub-set selection) can be adapted to the target tissue. It should be appreciated that the matrix array 615 can employ various combinations and patterns of needle electrodes 14, side-port injection needles 20, standard injection needles, and dual-purpose injection needle electrodes 625 (which can be side-port and/or standard injection types). It should also be appreciated that, when the matrix array 615 employs side-port injection needles and/or side-port dual purpose needle electrodes 625, their respective port arrays 25 can be oriented within the matrix array 615 to take advantage of the fluid dispersion mechanics within the target tissue. For example, the port arrays 25 can be oriented within the matrix array 615 in selected directions based on a planned array 615 insertion orientation within the tissue, as described in more detail below.

As shown in FIG. 35C, the matrix array 615 can be placed with respect to muscle tissue 675 so that the dual-purpose injection needle electrodes 625 are oriented as desired with respect to the muscle tissue, particularly with respect to the direction of muscle fiber extension M1. For example, the matrix array 615 can be oriented so that the longitudinal direction X1 of the array 615 extends along the direction of muscle fiber extension M1, as indicated by the array 615 position shown in dashed lines. Alternatively, the physician can elect to orient the array 615 so the longitudinal direction X1 is oriented substantially perpendicular to the direction of muscle fiber extension M1, which can therefore provide. At such a planned array 615 insertion orientation, the port arrays 25 of the side-port injection needles 20, 625 can be oriented so that their side-port flow directions 88 are oriented along the direction of muscle fiber extension M1. In this manner, the array 615 can span a greater number of individual muscle fiber striations and can direct the injectate along the greater number of individual muscle fiber striations. Such selective orientations and usages of the array 615 can be further tailored by the application of the pulsing pattern with respect to specific sub-sets of dual-purpose electrodes, which pulsing patterns can be adapted to focus the EP field along the direction of muscle fiber extension M1. These configurations and usages can also take advantage of the fact that, during EP electrical current flow, the impedance is reduced when directed in the same direction as the muscle fibers. Moreover, the direction 88 of fluid ejection from the injection needles 20, 625, when oriented along the muscle fiber striations, can also be expected to experience less mechanical impedance to fluid flow, which can allow for beneficial drug distribution along the electroporation field.

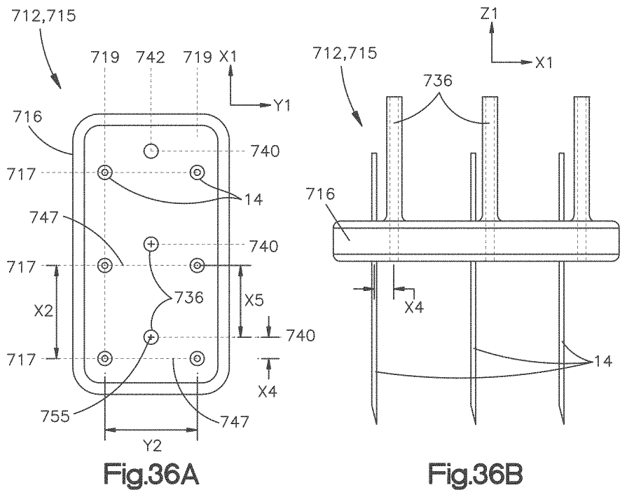

Referring now to FIGS. 36A-36B, an example embodiment of an array assembly 712 is shown having a matrix electrode array 715 coupled to a support member 716. In this example embodiment, the matrix array 715 includes a plurality of needle electrodes 14 arranged in rows 717 and columns 719 and having injection channels 736 located between the needle electrodes 14, generally similar to the embodiments described above with reference to FIGS. 30A-30B, FIGS. 31A-32B, FIG. 33, and FIGS. 35A-35C. However, in the present embodiment, one or more and up to all of the injection channels 736 is eccentrically offset from adjacent rows 717 and/or adjacent columns 719. As used herein with respect to an injection channel 736 and an adjacent row 717 and/or adjacent column 719, the phrase “eccentrically offset” means that the injection channel 736 is spaced from the nearest row 717 and/or column 719 along a respective direction and at a respective offset distance that is less than a distance along the respective direction between the injection channel 736 and the next nearest row 717 and/or column 719.

In the illustrated embodiment, each of the injection channels 736 is eccentrically offset from the respective nearest row 717 along the longitudinal direction X1. In particular, each injection channel 736 of the illustrated embodiment is longitudinally spaced from the nearest row 717 at an offset distance X4 that is less than a secondary offset distance X5 between the injection channel 736 and the next nearest row 717. The offset distance X4 and the secondary offset distance X5 are measured between the central axis 755 of the injection channel 736 and the nearest electrode row axis 747 and the next nearest electrode row axis 747, respectively. The offset distance X4 can be quantified as a factor (i.e., multiple) of the secondary offset distance X5. For example, the offset distance X4 can range from a factor of about 0.001 to a factor of about 0.999 of the secondary offset distance X5.

According to a non-limiting example of the illustrated embodiment, the matrix array 715 has six (6) electrodes 14 arranged in a 3×2 matrix (i.e., three (3) rows 717 and two (2) columns 719), with equidistant row and column spacing X2, Y2. The injection channels 736 are arranged in a 3×1 channel array (i.e., three (3) rows 740 and one column 742 of channels 136) such that each injection channel 736 is eccentrically offset from the nearest row 717 of electrodes 14 at equidistance offset distances X4. In this example, each offset distance X4 is a factor of about 0.25 of the respective secondary offset distance X5. In particular, in this example the electrode row spacing X2, electrode column spacing Y2, and the channel row spacing X3 are each about 10 mm, with the injection channels eccentrically offset at an offset distance X4 of about 2.5 mm along the longitudinal direction X1. It should be appreciated that any of these spacing distances X2, Y2, and offsets X4, X5 can be adjusted as needed.

It should also be appreciated that, in other embodiments, the injection channels 736 can be eccentrically offset from one of the electrode columns 719 along the lateral direction Y1. It should yet also be appreciated that the number of electrodes 14 and/or injection channels 736 in the matrix array 715 can be reduced or increased as needed based on various factors, such as the target treatment location, target tissue, and injection volume, by way of non-limiting examples. For example, the matrix array 715 can be increased to include one or more additional rows 717 and/or columns 719 of electrodes 14 and/or one or more additional rows 740 and/or columns 742 of injection channels 736, such that the injection channels 736 are eccentrically offset from the electrode rows 717. It should further be appreciated that the matrix array 715 can employ a combination of eccentrically offset injection channels 717 and injection channels 717 that are not eccentrically offset (such as by being located equidistantly between respective electrodes 14 or by being aligned with a respective electrode row 717).

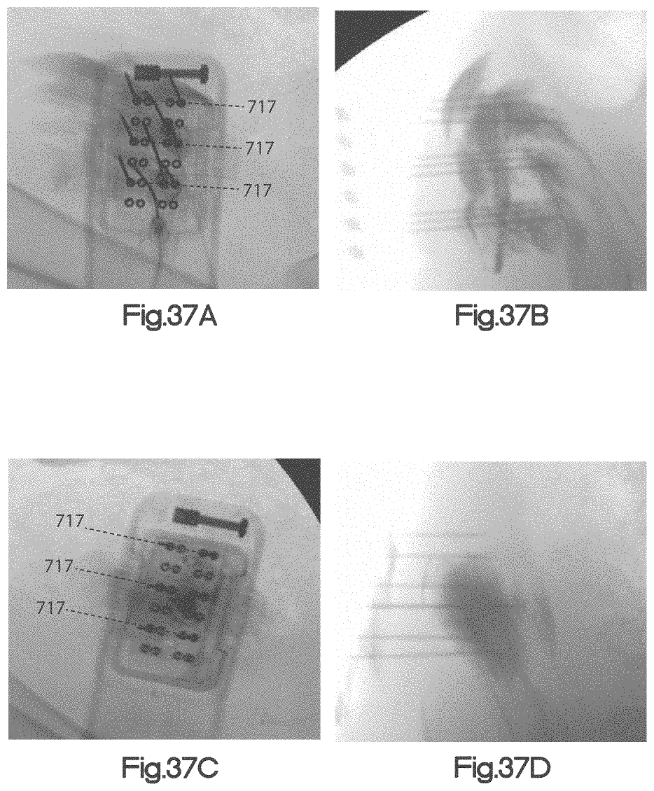

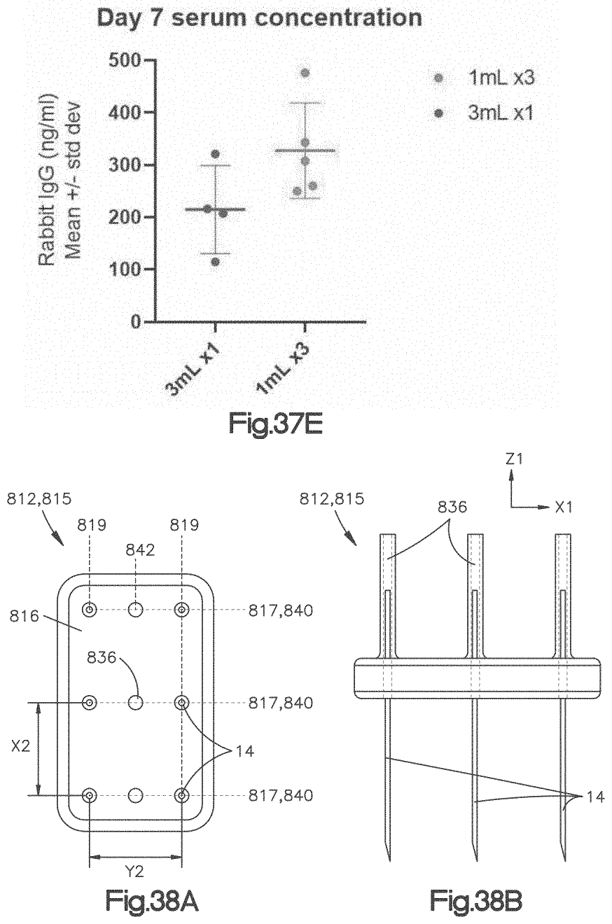

The present embodiment provides significant advantages for electroporation treatment combined with side-port injection. One advantage is that by employing multiple injection channels 736 within the electrode array 715, the agent dosage can be fractionated among multiple injection sites, resulting in enhanced fluid dispersion in target tissue. Referring now to FIGS. 37A-37D, it can be seen that fractionating an injection of 3 mL equally through three (3) separate injection channels 736 (i.e., 1 mL per injection channel 736) via side-port needle injection (FIGS. 37A-37B) provides superior fluid dispersion in muscle compared to a single-channel 736, 3-mL injection via side-port needle injection (FIGS. 37C-37D). The images shown in FIGS. 37A-37D were generated in live pig quadriceps muscles using fluoroscopy to visualize a radiocontrast agent injected via side-port injection needles in a matrix array having the configuration shown in FIGS. 36A-36B, with 10-mm spacings for X2, Y2, and X3 and a 2.5-mm offset distance X4. The single-channel injection shown in FIGS. 37C-37D was performed through the middle channel 736. In these images, the matrix array is oriented such that the electrode rows 717 extend substantially perpendicular to the direction of muscle fiber extension M1.

Referring now to FIG. 37E, comparative results demonstrate that utilizing multiple injection sites in the array, and targeting those injections with respective electroporation fields, enhanced dMAb expression in rabbits compared to single-injection delivery. This data was generated in rabbits via injection into quadriceps muscle using a matrix array configured as shown in FIGS. 37A-37B. The dMAb expression was measured in serum at 7 days post-delivery. The same electroporation parameters were applied to both groups.

Referring now to FIGS. 38A-38B, in another example embodiment, an array assembly 812 has a support member 816 that includes a matrix array 815 configured similar to the embodiment described above with reference to FIGS. 36A-36B. As with the aforementioned embodiment, the matrix array 815 has six (6) electrodes 14 arranged in a 3×2 matrix, with equidistant electrode row and column spacing X2, Y2, and three (3) injection channels 836 arranged in a 3×1 channel array. In the present embodiment, however, the injection channels 836 are aligned with the rows 817 of electrodes 14, such that the injection channels 836 are intersected by the respective electrode row axes 847. In one non-limiting example of the matrix array 815, the array 815 can employ an electrode row spacing X2, electrode column spacing Y2, and channel row spacing X3 that are each about 10 mm. It should be appreciated that any of these spacing distances X2, Y2, X3 can be adjusted as needed.

The matrix array 815 of the present embodiment provides significant advantages for electroporation treatment when combined with side-port injection. As with the matrix arrays described above, the array 815 employs multiple injection channels 836 that allows fractionating the agent dosage among multiple injection sites. Moreover, the dispersed injectate at the multiple injection sites can be targeted with respective electroporation fields delivered by respective subsets of electrodes 14 in the array 815. Another advantage is that the matrix array 815 can employ a pulse pattern that enhances co-localization of the electroporation fields with the side-port delivered fluid dispersions from the injection channels 836 aligned with the electrode rows 817. In particular, the matrix array 815 can employ a pulse pattern that delivers pulses between electrode pairs in each row 817, thereby directing the pulses across the area underneath the injection channels 836. This better co-localizes the electroporation fields with the fluid dispersions emanating from the side-port injection needles extending through the injection channels 836, as described in more detail below.

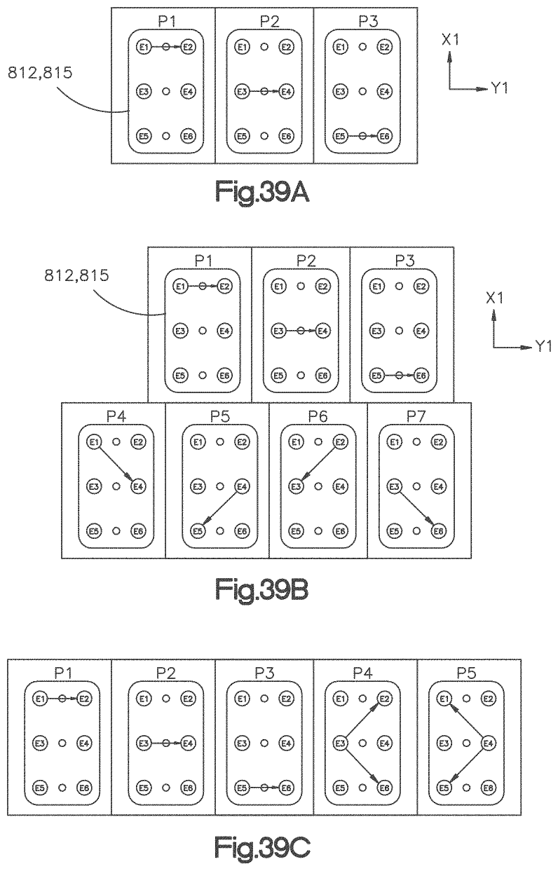

Referring now to FIG. 39A, an example pulse pattern will be described for the matrix array 815 shown in FIGS. 38A-38B. For purposes of illustrating the pulse pattern, the electrodes 14 of the matrix array 815 will be referred to by electrode positions E1-E6, in which electrode positions E1 and E2 are on a first electrode row 817, electrode positions E3 and E4 are on a second electrode row 817, and electrode positions E5 and E6 are on a third electrode row 817. In this example, the pulse pattern includes three (3) pulses, of which the first pulse P1 is delivered between E1 and E2, the second pulse P2 is delivered between E3 and E4, and the third pulse P3 is delivered between E5 and E6. In another example, the pulse pattern shown in FIG. 39A can be repeated, providing a pulse pattern having two identical pulse trains and a total of six (6) pulses. Such a repeated pulse pattern provides two pulses per electrode pair, which can facilitate enhanced electroporation results.

Referring now to FIG. 39B, in an additional example, a pulse pattern can employ the three pulses P1-P3 shown in FIG. 39A, plus four (4) additional pulses P4-P7 delivered diagonally between adjacent electrode rows 817 and columns 819. In this particular example, the fourth pulse P4 is delivered between E1 and E4, the fifth pulse P5 is delivered between E4 and E5, the sixth pulse P6 is delivered between E2 and E3, and the seventh pulse P7 is delivered between E3 and E6. The four (4) diagonal pulses P4-P7 can be beneficial for co-localizing the electroporation fields with any injectate that dispersed between the electrode rows 817 along the longitudinal direction X1.

Referring now to FIG. 39C, in a further example for co-localizing the electroporation fields with injectate that dispersed longitudinally between the electrode rows 817, a pulse pattern can effectively replace pulses P4-P7 shown in FIG. 39B with two (2) alternative pulses P4-P5 that each split the current diagonally from the center row 817 to the first and third rows 817. In particular, in this example the fourth pulse P5 is delivered from E3 to both E2 and D6, and the fifth pulse P5 is delivered from E4 to both E1 and E5. This pulse pattern can effectively target injectate dispersed between the electrode rows 817 using fewer total pulses than the pattern shown in FIG. 39B.

It should be appreciated that the example pulse patterns described above with reference to FIGS. 39A-39C represent non-limiting examples of pulse patterns that can be employed with the matrix array 815. It should also be appreciated that the foregoing pulse patterns can also be employed with the matrix array 715 shown in FIGS. 36A-36B. Furthermore, these pulse patterns can be adjusted as needed based on the particular factors involved.

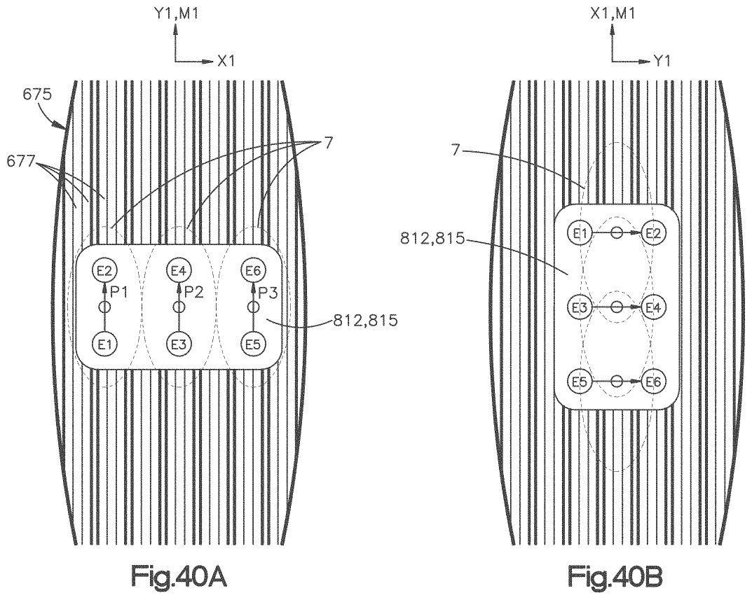

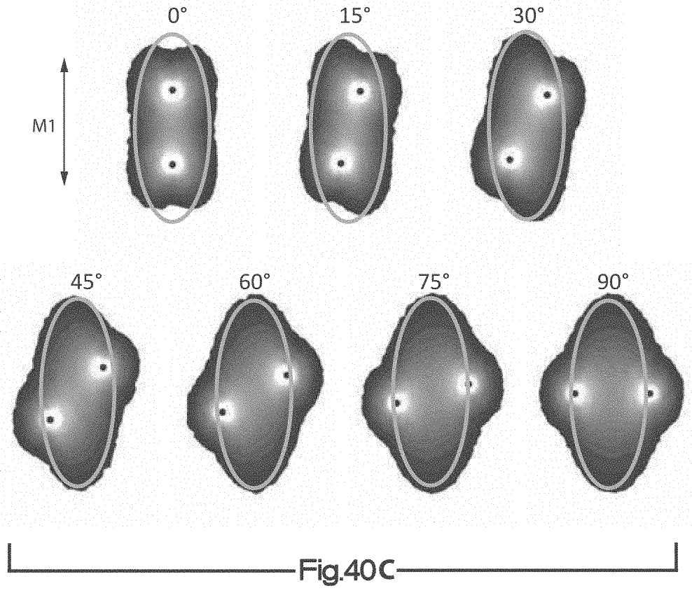

Referring now to FIG. 40A-40C, an additional advantage of the matrix array 815 described above with reference to FIGS. 38A-38B involves its particular effectiveness in tissues that influence fluid dispersion along specific directions. One such tissue is muscle tissue 675. As described above, intramuscular (IM) tissue tends to influence injected fluid 7 (e.g., the injectate) to disperse predominantly along the direction of muscle fiber extension M1. One particular advantage of the matrix array 815 is that its design allows favorable IM electroporation results regardless of its orientation relative to the direction of muscle fiber extension M1. In this manner, the matrix array 815 can be said to be more robust against mis-orientation in muscle.

As shown in FIG. 40A, the matrix array 815 can be inserted into muscle tissue 675 at an orientation whereby the electrode rows 817 align with the direction of muscle fiber extension M1. This orientation can be characterized as a “parallel” or “0-degree” orientation. In this orientation, each electrode row 817 and the associated injection channel 836 generally extends alongside and/or in-between the same muscle fibers 677. The three (3) fluid injections (utilizing the injection channels 836) disperse predominantly along the direction of muscle fiber extension M1, resulting generally in three side-by-side fluid dispersions 7. In this manner, each of electroporation pulses P1-P3 can effectively target the respective fluid dispersion 7 so that the high-magnitude portions of the electroporation fields co-localize with the respective fluid dispersions 7.

As shown in FIG. 40B, the matrix array 815 can alternatively be inserted into muscle tissue 675 at an orientation whereby the electrode rows 817 are oriented perpendicular to the direction of muscle fiber extension M1. This orientation can be characterized as a “perpendicular” or “90-degree” orientation. In this orientation, each electrode row 817 can traverse multiple muscle fibers 677. The three (3) fluid injections (utilizing the injection channels 836) disperse predominantly along the direction of muscle fiber extension M1, resulting generally in longitudinally overlapping fluid dispersions 7 having a maximum concentration between electrodes E3 and E4. In this manner, electroporation pulses P1-P3 can effectively target more muscle fibers and encompass more of the injected fluid than at the 0-degree orientation. Thus, a physician can employ the matrix array 815 at the 90-degree orientation to target more injectate with a more homogeneous electrical field, which can lead to transfecting more myocyte cells.

Referring now to FIG. 40C, each electrode pair (i.e., the electrodes in a single row 817) demonstrate strong co-localization of the electroporation field and the fluid dispersion regardless of the array orientation relative to the direction of muscle fiber extension M1. For example, at the 0-degree orientation, the high-magnitude portion of the electrical field aligns with the high-concentration portion of the fluid dispersion 7. One reason for this result is because the muscle fibers 677 demonstrate anisotropic electrical conductivity that is highest along the direction of muscle fiber extension M1. Thus, electrical impedance is minimized along direction M1. Additionally, muscle fibers provide a lower mechanical fluid impedance along the direction of muscle fiber extension M1, as discussed above. However, even when the orientation rotates toward higher angles, the injectate still disperses along the direction of muscle fiber extension M1 while the electrical field deforms (due to electrical conductivity being anisotropic and highest along the fiber axis) to somewhat match. Even at a 90-degree orientation, the electrical field is effectively “stretched” in direction M1, resulting in an electrical field that bulges out in the middle, where injectate is located. Thus, regardless of the array 815 orientation relative to muscle fibers, the array 815 beneficially co-localizes the electrical field with the injectate.

In other embodiments of the matrix array 815, the number of electrode rows 817 and/or columns 819 and/or the number of injection channel rows 840 and/or columns 842 of the matrix array 815 can be reduced or increased as needed based on various factors, such as the target treatment location, target tissue, and injection volume, by way of non-limiting examples. For example, the matrix array 815 can be increased to include one or more additional rows 817 and/or columns 819 of electrodes and/or one or more additional rows 840 and/or columns 842 of injection channels 836, such that the rows 840 of injection channels 836 are aligned with the rows 817 of electrodes 14. It should also be appreciated that the matrix array 815 can employ a combination of one or more injection channels 836 that are aligned with respective electrode rows 817 and one or more injection channels 836 that are offset from respective electrode rows 817 (including eccentrically offset or equidistantly offset).

It should be appreciated that the various parameters of the side-port injection needles 20 and associated electrode arrays 15, 215, 315, 415, 515, 615, 715, 815 described above are provided as exemplary features, such as for enhancing co-localization of injectates within an electroporation field and thereby enhancing electroporative transfection. These parameters can be adjusted as needed without departing from the scope of the present disclosure.

It should be understood that when a numerical preposition (e.g., “first”, “second”, “third”) is used herein with reference to an element, component, dimension, or a feature thereof (e.g., “first” electrode, “second” electrode, “third” electrode), such numerical preposition is used to distinguish said element, component, dimension, and/or feature from another such element, component, dimension and/or feature, and is not to be limited to the specific numerical preposition used in that instance. For example, a “first” electrode, direction, or support member, by way of non-limiting examples, can also be referred to as a “second” electrode, direction, or support member in a different context without departing from the scope of the present disclosure, so long as said elements, components, dimensions and/or features remain properly distinguished in the context in which the numerical prepositions are used.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. In particular, one or more of the features from the foregoing embodiments can be employed in other embodiments herein. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. An injection device for in vivo delivery of an agent, comprising:
a tubular body defining a lumen that extends along a central axis oriented along a longitudinal direction, wherein a distal end of the lumen is occluded, the tubular body defining at least one side-port extending from the lumen to an outer surface of the tubular body, wherein the at least one side-port is elongated along the outer surface of the tubular body.

2. The injection device of claim 1, wherein the at least one side-port has a length and a width each measured along the outer surface, wherein the length is greater than the width by a factor in a range of about 2 to about 80.

3. The injection device of claim 2, wherein the factor is in a range of about 15 to about 50.

4. The injection device of claim 3, wherein the factor is in a range of about 35 to about 45.

5. The injection device of claim 2, wherein the at least one side-port comprises a plurality of side-ports.

6. The injection device of claim 5, wherein the plurality of side-ports are arranged into distinct rows spaced from each other along the longitudinal direction.

7. The injection device of claim 6, wherein at least a first one of the rows comprises four side-ports of the plurality of side-ports, and wherein the four side-ports are evenly angularly spaced from each other about a circumference of the tubular body.

8. The injection device of claim 7, wherein at least a second one of the rows comprises four additional side-ports of the plurality of side-ports, the four additional side-ports being evenly angularly spaced from each other about a circumference of the tubular body, and wherein the four additional side-ports are angularly offset from the four side-ports of the at least first one of the rows by an offset angle of about 45 degrees about the central axis.

9. The injection device of claim 8, wherein the four side-ports and the four additional side-ports are each elongated along the longitudinal direction.

10. The injection device of claim 8, wherein the four side-ports and the four additional side-ports are each rectangular.

11. The injection device of claim 8, wherein at least a third one of the rows comprises four yet additional side-ports of the plurality of side-ports, the four yet additional side-ports being evenly angularly spaced from each other about a circumference of the tubular body, and wherein the four yet additional side-ports are angularly aligned from the four side-ports of the at least first one of the rows.

12. The injection device of claim 11, wherein the plurality of side-ports are bounded within a region of the tubular body, wherein the region has a length in a range of about 3.0 mm to about 12.0 mm.

13. The injection device of claim 12, wherein the length of the region is in a range of about 4.0 mm to about 6.0 mm.

14. The injection device of claim 1, wherein the injection device is an injection needle.

15. The injection device of claim 14, wherein the distal end of the lumen is occluded by a plug.

16. The injection device of claim 15, wherein the plug is constructed of a metallic material, and the plug is laser-welded to a distal portion of an interior surface of the injection needle within the lumen.

17. The injection needle of claim 15, wherein the plug is constructed of a metallic material, the plug defines a bevel of the needle, the plug has an insertion portion configured for insertion within a distal end of the injection needle, and the plug is laser-welded to the distal end of the injection needle.

18. The injection device of claim 15, wherein the plug is constructed of a polymeric material, and the plug is bonded to a distal portion of an interior surface of the injection needle within the lumen.

19. The injection device of claim 14, wherein the injection needle has a proximal end that defines a proximal bevel configured to pierce a drug cartridge.

20. An assembly for in vivo delivery of an agent, comprising:
an electroporation device having an electrode array that includes a plurality of needle electrodes configured for delivering one or more electroporation pulses to tissue;
at least one injection needle attachable to the electroporation device so as to extend substantially parallel with at least one of the plurality of needle electrodes, the at least one injection needle defining a lumen that extends along a central axis oriented along a longitudinal direction, wherein a distal end of the lumen is occluded, the at least one injection needle defining at least one side-port extending from the lumen to an outer surface of the at least one injection needle, wherein the at least one side-port is elongated along the outer surface of the injection needle.

21. The assembly of claim 20, wherein the at least one side-port comprises a plurality of side-ports, and the plurality of side-ports are configured to disperse injectate through at least one of muscle tissue and adipose tissue.

22. The assembly of claim 22, wherein the plurality of side-ports are configured to disperse injectate through both muscle tissue and adipose tissue.

23. The assembly of claim 20, wherein the at least one injection needle is located intermediate the plurality of needle electrodes, and the at least one side-port is configured to eject fluid from the lumen into tissue intermediate the plurality of needle electrodes.

24. The assembly of claim 23, wherein the plurality of needle electrodes are carried by a support member that also defines at least one injection channel for receiving the at least one injection needle.

25. The assembly of claim 24, wherein the plurality of needle electrodes are arranged in a circular pattern, and the at least one injection needle is centrally disposed in the circular pattern when attached to the electroporation device.

26. The assembly of claim 24, wherein the plurality of needle electrodes are arranged in a matrix having two or more rows and two or more columns of the needle electrodes.

27. The assembly of claim 26, wherein the matrix has three or more rows and two or more columns, and the support member defines at least three injection channels.

28. The assembly of claim 27, wherein the support member has circuitry providing electrical communication to each of the plurality of needle electrodes individually, such that select subsets of needle electrodes are configured to deliver electroporation pulses.

29. The assembly of claim 20, wherein each of the plurality of needle electrodes is a side-port injection needle configured to both deliver injectate to the target tissue and deliver the one or more electroporation pulses to the tissue.

30. An electroporation system for causing in vivo reversible electroporation in cells of tissue, comprising:
an electrode array that includes:
a support member having a top surface and a bottom surface, the support member defining a plurality of channels extending from the top surface to the bottom surface;
a plurality of needle electrodes coupled to the support member and extending through the plurality of channels, such that distal ends of the plurality of needle electrodes extend to a needle depth below the bottom surface of the support member, wherein the plurality of needle electrodes are arranged in a pattern along the support member, wherein at least some of the plurality of needle electrodes are dual-purpose injection needle electrodes configured to inject an agent into the tissue and to deliver one or more electroporation pulses to the tissue for causing the reversible electroporation in the cells of the tissue.

31. The electroporation system of claim 30, further comprising an applicator having a handle and a mounting portion connected to the handle, wherein the electrode array is attachable to the mounting formation, and the plurality of needle electrodes are in communication with circuitry of the applicator for controlling delivery of one or more electroporative pulses to the plurality of needle electrodes.

32. The electroporation system of claim 31, further comprising tubing connected to and in fluid communication with the dual-purpose injection needle electrodes, wherein the tubing is configured for delivering injectate from a reservoir assembly to the dual-purpose injection needle electrodes.

33. The electroporation system of claim 32, wherein all of the plurality of needle electrodes are dual-purpose injection needle electrodes.
